(19) United States
(12) Patent Application Publication
(10) Pub. No.: US 2009/0170790 A1
(43) Pub. Date: Jul. 2, 2009

(54) KETOLIDE DERIVATIVES AS ANTIBACTERIAL AGENTS

(76) Inventors: Biswajit Das, Gurgaon (IN); Mohammad Salman, Princeton, NJ (US); Santosh Haribhau Kurhade, Pune (IN); Ramadass Venkataramanan, New Delhi (IN); Rajesh Kumar, Rohini (IN); Gobind Singh Kapkoti, Navi Mumbai (IN); Rita Katoch, Chandigarh (IN); Anish Bandyopadhyay, Gurgaon (IN); Ashok Rattan, New Delhi (IN)

Correspondence Address: RANBAXY INC., 600 COLLEGE ROAD EAST, SUITE 2100, PRINCETON, NJ, 08540, US

(21) Appl. No.: 11/577,900

(22) PCT Filed: Oct. 25, 2005
(86) PCT No.: PCT/IB05/03181

§ 371 Date: Mar. 4, 2009

(30) Foreign Application Priority Data
Oct. 25, 2004 (IN) 2085/DEL/2004

(51) Int. Cl. A61K 31/7028; C07H 17/08; A61P 31/04

(52) U.S. Cl. 514/28; 536/7.1

(57) ABSTRACT

The present invention provides ketolide derivatives, which can be used as anti-bacterial agents. Compounds disclosed herein can be used for the treating or preventing conditions caused by or contributed to by gram positive, gram negative or anaerobic bacteria, more particularly against, for example, Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus, Enterobactericeae or any combination thereof. Also provided are processes for preparing compounds disclosed herein, intermediates used in their synthesis, pharmaceutical compositions thereof, and methods of treating bacterial infections.

### FIELD OF THE INVENTION

The present invention provides ketolide derivatives, which can be used as anti-bacterial agents. Compounds disclosed herein can be used for the treating or preventing conditions caused by or contributed to by gram positive, gram negative or anaerobic bacteria, more particularly against, for example, Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus, Enterobactericeae or any combination thereof. Also provided are processes for preparing compounds disclosed herein, intermediates used in their synthesis, pharmaceutical compositions thereof, and methods of treating bacterial infections.

### BACKGROUND OF THE INVENTION

First generation macrolides, such as erythromycin A and early derivatives, can be characterized by bacteriostatic or bactericidal activity for most gram-positive bacteria, atypical pathogens and many community-acquired respiratory infections, particularly in patients with penicillin allergies. However, erythromycin A causes numerous drug-drug interactions, has relatively poor absorption, poor local tolerance, loses its antibacterial activity under acidic conditions by degradation and the degraded products are known to cause undesired side effects (Itoh, Z et al., Am. J. Physiol, 1984, 247:688; Omura, S et al., J. Med. Chem., 1987, 30:1943). Various erythromycin A derivatives have been prepared to overcome the acid instability and other problems associated with it.

Roxithromycin, clarithromycin and azithromycin were developed to address the limitations of erythromycin A. Both clarithromycin and azithromycin are reportedly important drugs in the treatment and prophylaxis of atypical mycobacterial infections in patients with HIV.

Macrolides are reportedly effective drugs in the treatment of many respiratory tract infections. However, increasing resistance among S. pneumoniae has prompted the search for new compounds that retain favorable safety profiles, retain a spectrum of activity and are confined to respiratory pathogens. Consequently, numerous investigators have prepared chemical derivatives of erythromycin A in an attempt to obtain analogs having modified or improved profiles of antibiotic activity. Ketolides exhibit greater efficacy and safety, have broader spectrum of activities, and are particularly effective against resistant pathogens; hence, ketolides have been developed as next generation macrolides.

U.S. Pat. No. 5,635,485 discloses erythromycin compounds that are reportedly useful in the treatment of bacterial infections in warm-blooded animals. U.S. Pat. No. 5,866,549 discloses novel semi-synthetic macrolides reportedly having antibacterial activity, more particularly, 6-O-substituted erythromycin ketolide derivatives and a method of treating bacterial infections. U.S. Pat. No. 6,472,372 discloses 6-O-carbamoyl ketolide antibacterials and a method of treating bacterial infections. PCT Publication No. WO 2004/029066 discloses bifunctional heterocyclic compounds that are reportedly useful as anti-infective, anti-proliferative, anti-inflammatory and prokinetic agents. PCT Publication No. WO 00/62783 discloses ketolide antibacterials that are reportedly useful in the treatment of bacterial and protozoal infections and in the treatment of other conditions involving gastric motility. PCT Publication No. WO 00/44761 discloses ketolide antibiotics reportedly useful as antibacterial and antiprotozoal agents in mammals. U.S. Pat. No. 5,747,467 discloses novel antibacterial composition and method of treating bacterial infections of gram positive bacteria in warm-blooded animals. U.S. Pat. No. 6,433,151 discloses demethylated ketolide derivatives and their use as medicament for the treatment of infection caused by gram positive bacteria, Haemophilus influenzae, Moraxalla spp. U.S. Pat. Nos. 6,458,771 and 6,399,582 disclose ketolide antibacterials that are reportedly useful in the treatment of bacterial and protozoal infections and in the treatment of other conditions involving gastric motility. U.S. Patent Application Nos. 2002/0115621 and 2003/0013665 disclose macrolide compounds that are reportedly useful as antibacterial and antiprotozoal agents in mammals, including man, as well in fish and birds. European Patent No. 1 114 826 discloses macrolide compounds that are reportedly useful antibacterial, antiprotozoal and/or prokinetic agents, also, it relates to a method of treating cancer or atherosclerosis. U.S. Pat. Nos. 6,313,101 and 6,407,257 disclose derivatives of erythromycin that reportedly have good antibiotic activity on gram-positive bacteria. Other ketolide compounds have also been reported. A. Denis and A. Bonnefoy, Drugs of the Future, 26(10):975-84 (2001), Champney W. S., et al., Current Microbiology, 42 :203-10 (2001).

However, there remains a need for novel ketolide derivatives that are useful in treating or preventing bacterial infections.

### SUMMARY OF THE INVENTION

The present invention provides ketolide derivatives, which can be used in the treatment or prevention of bacterial infections, and processes for the synthesis of these compounds.

Pharmaceutically acceptable salts, pharmaceutically acceptable solvates, stereoisomers, metabolites, prodrugs, polymorphs of these compounds having same type of activity are also provided.

Pharmaceutical compositions containing the disclosed compounds together with pharmaceutically acceptable carriers, excipients or diluents, which can be used for the treatment of bacterial infection.

Thus in one aspect, provided herein are compounds having the structure of Formula I,

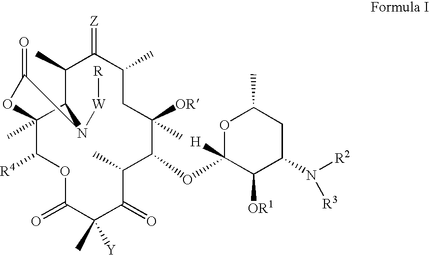

pharmaceutically acceptable salts, pharmaceutically acceptable solvates, stereoisomers, prodrugs, metabolites and polymorphs thereof, wherein:
R¹ can be hydrogen, hydroxyl protecting group;
R² and R³ can independently be hydrogen, alkyl, alkenyl, alkynyl, cycloalkyl, aryl, (heterocyclyl)alkyl or —COR¹¹, wherein
   R¹¹ can be hydrogen, alkyl, aryl, NR⁹R¹⁰ or alkoxy;
   R⁹ and R¹⁰ can independently be hydrogen, alkyl, alkenyl or alkynyl; and
   with the proviso that R² and R³ are not simultaneously methyl;
W can be —NH or —(CH₂)ₘ—, wherein
   m can be an integer of from 2 to 6;
   —(CH₂)ₘ— group can optionally be interrupted by one or more of unsaturated bond, oxygen, sulfur, —NRₐ— or combination thereof, wherein
      Rₐ can be hydrogen, alkyl, cycloalkyl, alkenyl, heterocyclyl, (heterocyclyl)alkyl, alkynyl or aryl;
   one of the hydrogen atoms of —(CH₂)ₘ— group can optionally be replaced by halogen, alkyl, hydroxyl or alkoxy; and
   with the proviso that R² is hydrogen when W is —(CH₂)ₘ—, wherein m is an integer of from 2 to 6;
R can be hydrogen, hydroxy, alkyl, aryl, heterocyclyl, cycloalkyl or cycloalkenyl;
R⁴ can be hydrogen, alkyl, alkenyl, alkynyl, aryl, cycloalkyl, heterocyclyl, amidoarylalkyl or amidoarylalkynyl;
R′ can be hydrogen, aryl, alkyl or —(CH₂)ᵣ-U; wherein
   r can be an integer of from 1 to 4; and
   U can be alkenyl, alkynyl, alkylalkenyl, alkylalkynyl, arylalkenyl, arylalkynyl, heterocyclylalkenyl or heterocyclylalkynyl; or
   OR′ can be replaced by hydrogen;
Y can be hydrogen, halogen, cyano, alkyl, aryl, heterocyclyl, hydroxy, amino, PhSe, alkenyl, alkynyl or —NR⁹R¹⁰, wherein
   R⁹ and R¹⁰ are the same as defined earlier; and
Z can be oxygen, sulphur or NOR¹¹, wherein
   R¹¹ is the same as defined earlier.

The compounds described herein can include one or more of the following embodiments. For example, R¹ can be hydrogen; R² can be hydrogen or alkyl; R⁴ can be alkyl; Y can be halogen; R³ can be alkyl or alkenyl; R′ can be alkyl; Z can be oxygen or NOR¹¹; W can be —NH or —(CH₂)₄—, wherein the —(CH₂)₄— group can be interrupted by oxygen, nitrogen or unsaturated bond or one of the hydrogen atoms of —(CH₂)₄— group can be replaced by alkyl; and R is hydrogen, aryl, substituted aryl or heterocyclyl. In another example, R¹ can be hydrogen; R² can be hydrogen or methyl; R⁴ can be ethyl; Y can be fluorine; R³ can be ethyl or allyl; R′ can be methyl; Z can be oxygen or —NOCH₃; W can be —NH—, —(CH₂)₃O—, —NH—(CH₂)₃—, —NHCH₂CH═CH— or —NH(CH₂)₂—CH(CH₃)— and R can be phenyl, 3-(pyridine-3-yl)-phenyl, 3-(thienyl-3-yl)-phenyl, pyridin-3-yl, imidazo[4,5-b]pyridin-3-yl, pyrrolo[2,3b]pyridin-1-yl, isoquinoline-5-yl and benzimidazol-1-yl, 3-(1H-imidazol-4-yl)-pyridine, 4-phenyl-1H-imidazole or 4-thiophen-3-yl-1H-imidazole.

In another aspect, provided herein are compounds selected from:
2-α-Fluoro-5-O-(3′-N-didesmethyl-3′-N-ethyl)-11,12-dideoxy-3-O-decladinosyl-6-O-methyl-3-oxo-12,11-[oxycarbonyl-((4-imidazol[4,5-b]pyridin-3-yl)-butyl)-imino]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(3-pyridin-3-yl-phenoxy)-propyl)-imino)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(3-pyridin-3-yl-phenoxy)-propyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(pyridin-3-yloxy)-propyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(3-thiophen-3-yl-phenoxy)-propyl)-imino)]erythromycin A,
2-α-Fluoro-5-O-(3-N-desmethyl-3-N-ethyl)-11,12-dideoxy-3-O-decladinosyl-6-O-methyl-3-oxo-12,11-[oxycarbonyl-hydrazo]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-hydrazo]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-pyrrolo[2,3-b]pyridin-1-yl)-propyl)-hydrazo)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-phenylpropyl)-hydrazo)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-isoquinolin-5-yl-propyl-hydrazo)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-phenylpropyl)-hydrazo)]erythromycin A,
2-α-Fluoro-5-O-(3′-N-desmethyl-3′-N-allyl)-11,12-dideoxy-3-O-decladinosyl-6-β-methyl-3-oxo-12,11-[oxycarbonyl-((3-(4-phenyl-imidazol-1-yl)-propyl)-hydrazo)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-phenyl-imidazol-1-yl)-propyl)-hydrazo)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-benzoimidazol-1-yl)-propyl)-hydrazo)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-pyridyl-3-yl-imidazol-1-yl)-propyl)-hydrazo)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-pyridyl-3-yl-imidazol-1-yl)-propyl)-hydrazo)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-benzoimidazol-1-yl)-propyl)-hydrazo)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-thiophen-3-yl-imidazol-1-yl)propyl)-hydrazo)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-phenyl-imidazol-1-yl)-propyl)-hydrazo)]erythromycin A-9-(O-methyl)oxime,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-pyridyl-3-yl-imidazol-1-yl)-propyl)-hydrazo)]erythromycin A-9-(O-methyl)oxime,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-thiophen-3-yl-imidazol-1-yl)propyl)-hydrazo)]erythromycin A-9-(O-methyl)oxime,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-pyridyl-3-yl-imidazol-1-yl)-butyl)-hydrazo)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-phenyl-imidazol-1-yl)-butyl)-hydrazo)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-phenyl-allyl)hydrazo)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-(2-chloro-pyrimidin-5-yl)-imidazol-1-yl)-propyl)-hydrazo)]erythromycin A, or
   pharmaceutically acceptable salts, pharmaceutically acceptable solvates, stereoisomers, prodrugs, metabolites or polymorphs thereof.

In yet another aspect, provided herein are pharmaceutical compositions comprising therapeutically effective amounts of one or more compounds having the structure of Formula I,

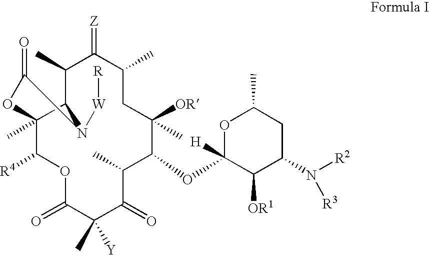

pharmaceutically acceptable salts, pharmaceutically acceptable solvates, stereoisomers, prodrugs, metabolites or polymorphs thereof, and optionally together with one or more pharmaceutically acceptable carriers, excipients or diluents,
wherein:
R¹ can be hydrogen, hydroxyl protecting group;
R² and R³ can independently be hydrogen, alkyl, alkenyl, alkynyl, cycloalkyl, aryl, (heterocyclyl)alkyl or —COR¹¹, wherein
   R¹¹ can be hydrogen, alkyl, aryl, NR⁹R¹⁰ or alkoxy;
   R⁹ and R¹⁰ can independently be hydrogen, alkyl, alkenyl or alkynyl; and
   with the proviso that R² and R³ are not simultaneously methyl;
W can be —NH or —(CH₂)ₘ—, wherein
   m can be an integer of from 2 to 6;
   —(CH₂)ₘ— group can optionally be interrupted by one or more of unsaturated bond, oxygen, sulfur, —NRₐ— or combination thereof, wherein
      Rₐ can be hydrogen, alkyl, cycloalkyl, alkenyl, heterocyclyl, (heterocyclyl)alkyl, alkynyl or aryl;
   one of the hydrogen atoms of —(CH₂)ₘ— group can optionally be replaced by halogen, alkyl, hydroxyl or alkoxy; and
   with the proviso that R² is hydrogen when W is —(CH₂)ₘ—, wherein m is an integer of from 2 to 6;
R can be hydrogen, hydroxy, alkyl, aryl, heterocyclyl, cycloalkyl or cycloalkenyl;
R⁴ can be hydrogen, alkyl, alkenyl, alkynyl, aryl, cycloalkyl, heterocyclyl, amidoarylalkyl or amidoarylalkynyl; Z
R′ can be hydrogen, aryl, alkyl or —(CH₂)ᵣ-U; wherein
   r can be an integer of from 1 to 4; and
   U can be alkenyl, alkynyl, alkylalkenyl, alkylalkynyl, arylalkenyl, arylalkynyl, heterocyclylalkenyl or heterocyclylalkynyl; or
   OR′ can be replaced by hydrogen;
Y can be hydrogen, halogen, cyano, alkyl, aryl, heterocyclyl, hydroxy, amino, PhSe, alkenyl, alkynyl or —NR⁹R¹⁰, wherein
   R⁹ and R¹⁰ are the same as defined earlier; and
Z can be oxygen, sulphur or NOR¹¹, wherein
   R¹¹ is the same as defined earlier.

In another aspect, provided are methods for treating or preventing a condition caused by or contributed to by bacterial infection in a mammal comprising administering to the mammal in need thereof a pharmaceutical composition described herein.

In yet another aspect, provided are methods for treating or preventing a condition caused by or contributed to by bacterial infection in a mammal comprising administering to the mammal in need thereof a therapeutically effective amount of one or more compounds having the structure of Formula I,

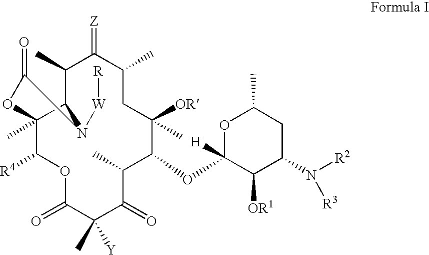

pharmaceutically acceptable salts, pharmaceutically acceptable solvates, stereoisomers, prodrugs, metabolites or polymorphs thereof, and optionally together with one or more pharmaceutically acceptable carriers, excipients or diluents,
wherein:
R¹ can be hydrogen, hydroxyl protecting group;
R² and R³ can independently be hydrogen, allyl, alkenyl, alkynyl, cycloalkyl, aryl, (heterocyclyl)alkyl or —COR¹¹, wherein
   R¹¹ can be hydrogen, alkyl, aryl, NR⁹R¹⁰ or alkoxy;
   R⁹ and R¹⁰ can independently be hydrogen, alkyl, alkenyl or alkynyl; and
   with the proviso that R² and R³ are not simultaneously methyl;
W can be —NH or —(CH₂)ₘ—, wherein
   m can be an integer of from 2 to 6;
   —(CH₂)ₘ— group can optionally be interrupted by one or more of unsaturated bond, oxygen, sulfur, —NRₐ— or combination thereof, wherein
      Rₐ can be hydrogen, alkyl, cycloalkyl, alkenyl, heterocyclyl, (heterocyclyl)alkyl, alkynyl or aryl;
   one of the hydrogen atoms of —(CH₂)ₘ— group can optionally be replaced by halogen, alkyl, hydroxyl or alkoxy; and
   with the proviso that R² is hydrogen when W is —(CH₂)ₘ, wherein m is an integer of from 2 to 6;
R can be hydrogen, hydroxy, alkyl, aryl, heterocyclyl, cycloalkyl or cycloalkenyl;
R⁴ can be hydrogen, alkyl, alkenyl, alkynyl, aryl, cycloalkyl, heterocyclyl, amido arylalkyl or amido arylalkynyl;
R′ can be hydrogen, aryl, alkyl or —(CH₂)ᵣ-U; wherein
   r can be an integer of from 1 to 4; and
   U can be alkenyl, alkynyl, alkylalkenyl, alkylalkynyl, arylalkenyl, arylalkynyl, heterocyclylalkenyl or heterocyclylalkynyl; or
   OR′ can be replaced by hydrogen;
Y can be hydrogen, halogen, cyano, alkyl, aryl, heterocyclyl, hydroxy, amino, PhSe, alkenyl, alkynyl or —NR⁹R¹⁰, wherein
   R⁹ and R¹⁰ are the same as defined earlier, and
Z can be oxygen, sulphur or NOR¹¹, wherein
   R¹¹ is the same as defined earlier.

Such methods can include one or more of the following embodiments. For example, the condition can be selected from community-acquired pneumonia, upper and lower respiratory tract infections, skin and soft tissue infections, hospital-acquired lung infections or bone and joint infections, mastitis, catheter infection, foreign body, prosthesis infections or peptic ulcer disease. The condition can also be caused by or contributed to by one or more gram positive, gram negative or anaerobic bacteria, wherein the one or more gram positive, gram negative or anaerobic bacteria are selected from Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus or Enterobactericeae. The one or more gram positive, gram negative or anaerobic bacteria can be a cocci and in other embodiments, the cocci can be drug resistant. The one or more compounds of Formula I can be concurrently or sequentially administered with one or more additional therapeutic agents selected from benzoyl peroxide, clindamycin, telithromycin, tretinoin, vitamin E, vitamin A and its derivatives, tetracycline, isotretinoin, vitamin C, vitamin D, chaparral, dandelion root, licoric root, Echinacea, kelp, cayenine, sassafras, elder flowers, pantothenic acid, para amino benzoic acid, biotin, cholin, inositol, folic acid, calcium, magnesium, potassium, vitamin B₆, zinc, carotenoid orazelaic acid or mixtures thereof.

In another aspect, provided are processes for preparing compounds of Formula XIII,

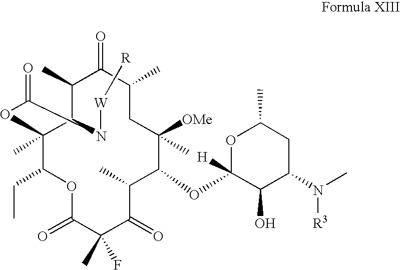

pharmaceutically acceptable salts, pharmaceutically acceptable solvates, enantiomers, diastereomers or polymorphs thereof, comprising the steps of:
(a) hydrolyzing clarithromycin of Formula II,

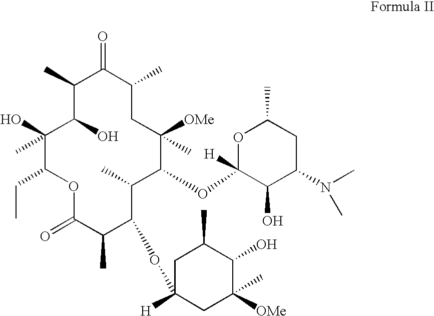

to form a compound of Formula III,

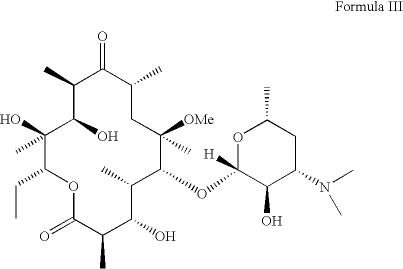

(b) protecting the compound of Formula III with one or more reagents of Formula R¹₂O or R¹X (wherein X is halogen) to form a compound of Formula IV,

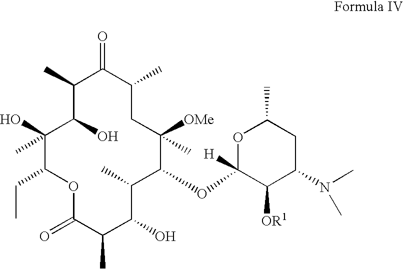

(c) reacting the compound of Formula IV with one or more suitable reagents to form a compound of Formula V,

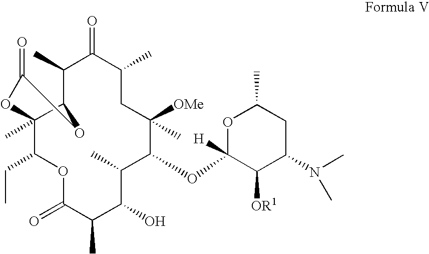

(d) reacting the compound of Formula V with one or more organic bases to form a compound of Formula VI,

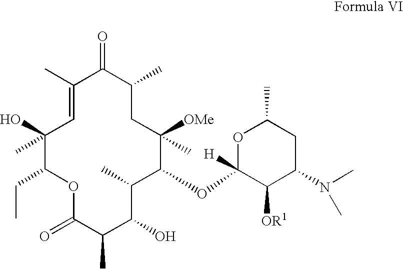

(e) oxidizing the compound of Formula VI to form a compound of Formula VII,

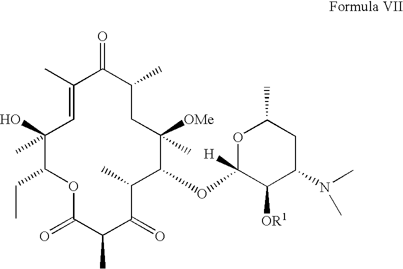

(f) desmethylating the compound of Formula VII at the 3′-N-dimethyl group to form a compound of Formula VIII,

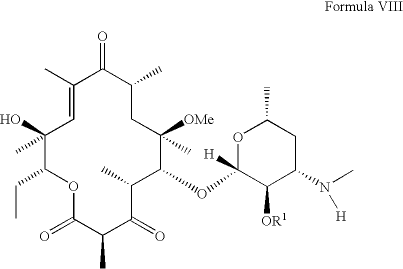

(g) alkylating the compound of Formula VIII with one or more reagents of Formula R³CHO, R³₂ CO or R³X to form a compound of Formula IX,

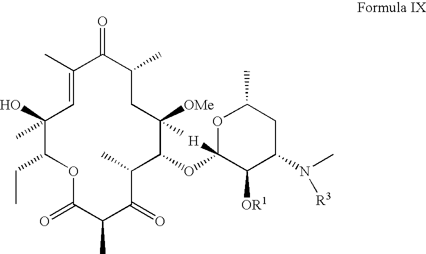

(h) fluorinating the compound of Formula IX to form a compound of Formula X,

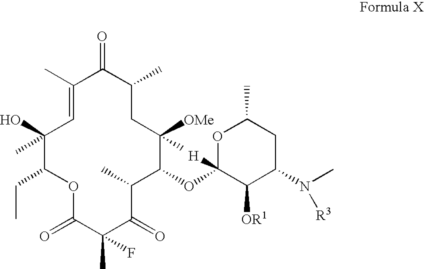

(i) reacting the compound of Formula X with N,N′-carbonyldiimidazole to form a compound of Formula XI,

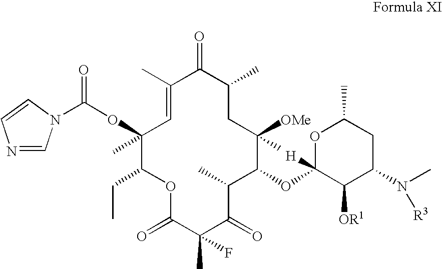

(j) reacting the compound of Formula XI with a compound of Formula R-W-NH₂ to form a compound of Formula XII,

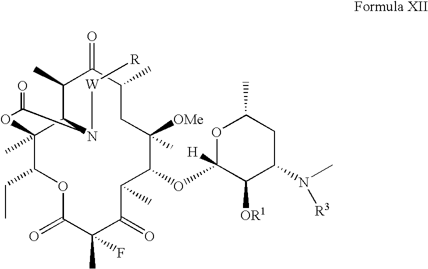

(k) deprotecting the compound of Formula XII to form a compound of Formula XIII (wherein R, R³, R¹ and W are the same as defined earlier),
wherein
R³ can be hydrogen, alkyl, alkenyl, alkynyl, cycloalkyl, aryl, (heterocyclyl)alkyl or —COR¹¹, wherein
   R¹¹ can be hydrogen, alkyl, aryl, NR⁹R¹⁰ or alkoxy;
   R⁹ and R¹⁰ can independently be hydrogen, alkyl, alkenyl or alkynyl; and
   with the proviso that R³ is not methyl;
W can be —NH or —(CH₂)ₘ—, wherein
   m can be an integer of from 2 to 6;
   —(CH₂)ₘ— group can optionally be interrupted by one or more of unsaturated bond, oxygen, sulfur, —NRₐ— or combination thereof, wherein
      Rₐ can be hydrogen, alkyl, cycloalkyl, alkenyl, heterocyclyl, (heterocyclyl)alkyl, alkynyl or aryl;
   one of the hydrogen atoms of —(CH₂)ₘ— group can optionally be replaced by halogen, alkyl, hydroxyl or alkoxy; and
R can be hydrogen, hydroxy, alkyl, aryl, heterocyclyl, cycloalkyl or cycloalkenyl; and
R¹ can be hydrogen, hydroxyl protecting group.

In yet another aspect, provided are processes for preparing compounds of Formula XIVA,

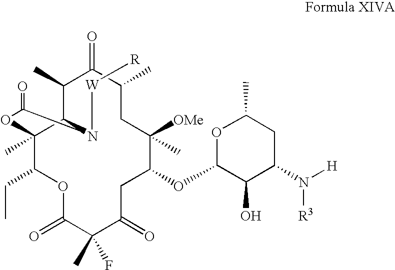

pharmaceutically acceptable salts, pharmaceutically acceptable solvates, stereoisomers, prodrugs, metabolites or polymorphs thereof comprising the steps of:
(a) desmethylating at 3′-N-dimethyl group of a compound of Formula XII

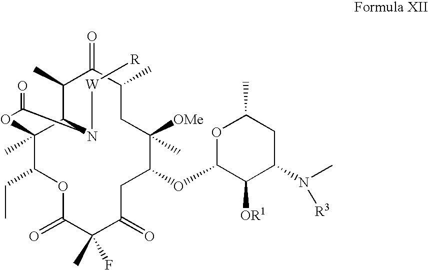

to form a compound of Formula XIIIA,

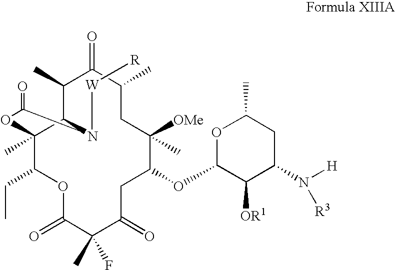

(b) deprotecting the compound of Formula XIIIA to form a compound of Formula XIVA,
wherein
R¹ can be hydrogen, hydroxyl protecting group;
R³ can be hydrogen, alkyl, alkenyl, alkynyl, cycloalkyl, aryl, (heterocyclyl)alkyl or —COR¹¹, wherein
   R¹¹ can be hydrogen, alkyl, aryl, NR⁹R¹⁰ or alkoxy;
   R⁹ and R¹⁰ can independently be hydrogen, alkyl, alkenyl or alkynyl; and
W can be —NH or —(CH₂)ₘ—, wherein
   m can be an integer of from 2 to 6;
   —(CH₂)ₘ— group can optionally be interrupted by one or more of unsaturated bond, oxygen, sulfur, —NRₐ— or combination thereof, wherein
      Rₐ can be hydrogen, alkyl, cycloalkyl, alkenyl, heterocyclyl, (heterocyclyl)alkyl, alkynyl or aryl;
   one of the hydrogen atoms of —(CH₂)ₘ— group can optionally be replaced by halogen, alkyl, hydroxyl or alkoxy; and
R can be hydrogen, hydroxy, alkyl, aryl, heterocyclyl, cycloalkyl or cycloalkenyl;

In another aspect, provided are processes for preparing compounds of Formula XVI,

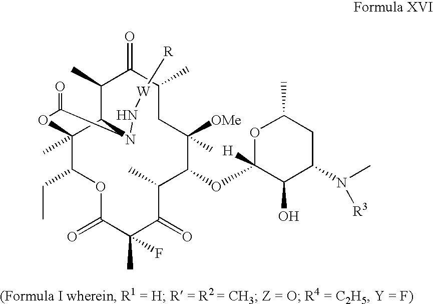

pharmaceutically acceptable salts, pharmaceutically acceptable solvates, stereoisomers, prodrugs, metabolites or polymorphs thereof, comprising the steps of:
(a) reacting the compound of Formula XI with hydrazine hydrate

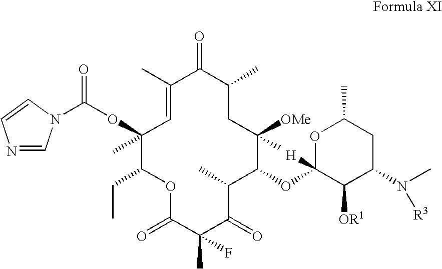

to form a compound of Formula XIV,

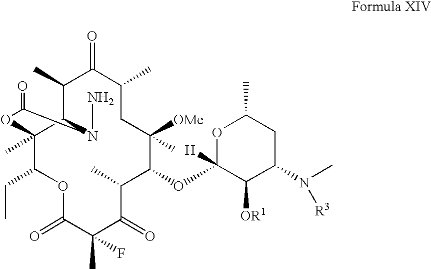

(b) deprotecting the compound of Formula XIV to form a compound of Formula XV, and

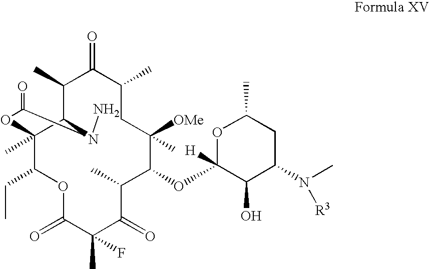

(c) reacting the compound of XV with a compound of Formula R-W-CHO to form a compound of Formula XVI,
wherein
R¹ can be hydrogen, hydroxyl protecting group;
R³ can be hydrogen, alkyl, alkenyl, alkynyl, cycloalkyl, aryl, (heterocyclyl)alkyl or —COR¹¹, wherein
   R¹¹ can be hydrogen, alkyl, aryl, NR⁹R¹⁰ or alkoxy;
   R⁹ and R¹⁰ can independently be hydrogen, alkyl, alkenyl or alkynyl; and
   with the proviso that R³ is not methyl;
W can be —NH or —(CH₂)ₘ—, wherein
   m can be an integer of from 2 to 6;
   —(CH₂)ₘ— group can optionally be interrupted by one or more of unsaturated bond, oxygen, sulfur, —NRₐ— or combination thereof, wherein
      Rₐ can be hydrogen, alkyl, cycloalkyl, alkenyl, heterocyclyl, (heterocyclyl)alkyl, alkynyl or aryl;
   one of the hydrogen atoms of —(CH₂)ₘ— group can optionally be replaced by halogen, alkyl, hydroxyl or alkoxy; and
R can be hydrogen, hydroxy, alkyl, aryl, heterocyclyl, cycloalkyl or cycloalkenyl;

In yet another aspect, provided are processes for preparing compounds of Formula XVIII,

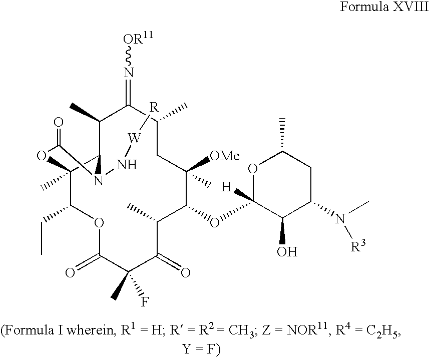

pharmaceutically acceptable salts, pharmaceutically acceptable solvates, enantiomers, diastereomers or polymorphs thereof comprising the steps of:
(a) reacting the compound of Formula XV with a compound of Formula H₂NOR¹¹

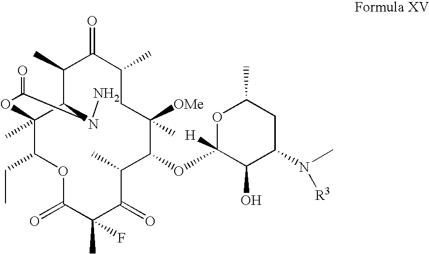

to form a compound of Formula XVII,

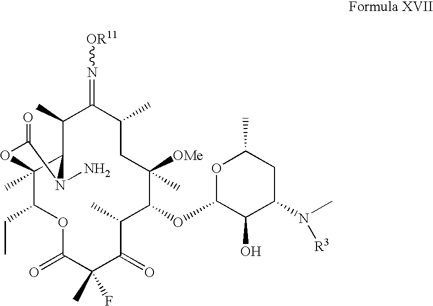

(b) reacting the compound of Formula XVII with a compound of Formula R—W—CHO to form a compound of Formula XVIII,
wherein
R¹¹ can be hydrogen, alkyl, aryl, NR⁹R¹⁰ or alkoxy, wherein
   R⁹ and R¹⁰ can independently be hydrogen, alkyl, alkenyl or alkynyl;
R³ can be hydrogen, alkyl, alkenyl, alkynyl, cycloalkyl, aryl, (heterocyclyl)alkyl or —COR¹¹, wherein
   R¹¹ can be the same as defined above; and
   with the proviso that R³ is not methyl;
W can be —NH or —(CH₂)ₘ—, wherein
   m can be an integer of from 2 to 6;
   —(CH₂)ₘ— group can be optionally interrupted by one or more of unsaturated bond, oxygen, sulfur, —NRₐ— or combination thereof, wherein
      Rₐ can be hydrogen, alkyl, cycloalkyl, alkenyl, heterocyclyl, (heterocyclyl)alkyl, alkynyl or aryl;
   one of the hydrogen atoms of —(CH₂)ₘ— group can optionally be replaced by halogen, alkyl, hydroxyl or alkoxy; and
R can be hydrogen, hydroxy, alkyl, aryl, heterocyclyl, cycloalkyl or cycloalkenyl.

In yet another aspect, provided herein are compounds of Formula I, which can also be used as anti-inflammatory and prokinetic agents.

### DETAILED DESCRIPTION OF THE INVENTION

In accordance with one aspect, provided herein are compounds having the structure of Formula I,

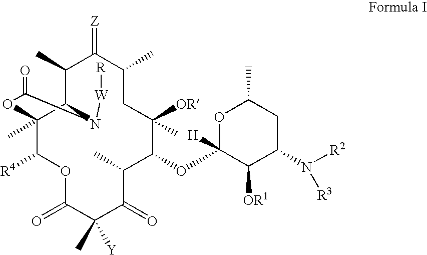

pharmaceutically acceptable salts, pharmaceutically acceptable solvates, stereoisomers, prodrugs, metabolites and polymorphs thereof, wherein:
R¹ can be hydrogen, hydroxyl protecting group;
R² and R³ can be independently hydrogen, alkyl, alkenyl, alkynyl, cycloalkyl, aryl, (heterocyclyl)alkyl or —COR¹¹, wherein
R¹¹ can be hydrogen, alkyl, aryl or NR⁹R¹⁰, wherein
   R⁹ and R¹⁰ are independently hydrogen, alkyl, alkenyl, alkynyl or alkoxy, with the proviso that R² and R³ are not simultaneously methyl;
      W can be —NH or —(CH₂)ₘ—, wherein
m can be an integer of from 2 to 6, and
—(CH₂)ₘ— group can be optionally interrupted by one or more of unsaturated bond, oxygen, sulfur, —NRₐ— or combination thereof, wherein
   Rₐ can be hydrogen, alkyl, cycloalkyl, alkenyl, heterocyclyl, (heterocyclyl)alkyl, alkynyl or aryl and
one of the hydrogen atom of —(CH₂)ₘ— group can be optionally replaced by halogen, alkyl, hydroxyl or alkoxy,
   with the proviso that R² is hydrogen when W is —(CH₂)ₘ—, wherein m can represent an integer of from 2 to 6;
   R can be hydrogen, hydroxy, alkyl, aryl, heterocyclyl, cycloalkyl, cycloalkenyl;
   R⁴ can be hydrogen, alkyl, alkenyl, alkynyl, aryl, cycloalkyl, heterocyclyl, amidoarylalkyl or amidoarylalkynyl;
   R′ can be hydrogen, aryl, alkyl or —(CH₂)ᵣ-U, wherein
r can be an integer of from 1 to 4, and
U can be alkenyl, alkynyl, alkylalkenyl, alkylalkynyl, arylalkenyl, arylalkynyl, heterocyclylalkenyl or heterocyclylalkynyl, or
OR′ can be replaced by hydrogen;
   Y can be hydrogen, halogen, cyano, alkyl, aryl, heterocyclyl, hydroxy, amino, PhSe, alkenyl, alkynyl or —NR⁹R¹⁰, wherein
R⁹ and R¹⁰ are the same as defined earlier; and
   Z can be oxygen, sulphur or NOR¹¹, wherein
R¹¹ is the same as defined earlier.

In one embodiment, R¹ can be hydrogen, R² can be hydrogen or alkyl; R⁴ can be alkyl; Y can be halogen, R³ can be alkyl or alkenyl; R′ can be alkyl; Z can be oxygen or NOR¹¹; W can be —NH or —(CH₂)₄—; —(CH₂)₄— group can be interrupted by oxygen, nitrogen or unsaturated bond or one of the hydrogen atom of —(CH₂)₄— group can be replaced by alkyl; R can be hydrogen, aryl, substituted aryl or heterocyclyl.

In another embodiment, R¹ can be hydrogen; R² can be hydrogen or methyl; R⁴ can be ethyl; Y can be fluorine; R³ can be ethyl or allyl; R′ can be methyl; Z can be oxygen or —NOCH₃; W can be —NH—, —(CH₂)₃O—, —NH—(CH₂)₃—, —NHCH₂CH═CH— or —NH(CH₂)₂—CH(CH₃)—; R can be phenyl, 3-(pyridine-3-yl)-phenyl, 3-(thienyl-3-yl)-phenyl, pyridin-3-yl, imidazo[4,5-b]pyridin-3-yl, pyrrolo[2,3b]pyridin-1-yl, isoquinolin-5-yl and benzimidazole-1-yl, 3-(1H-imidazol-4-yl)-pyridine, 4-phenyl-1H-imidazole or 4-thiophen-3-yl-1H-imidazole.

In accordance with a second aspect, provided herein are methods for treating or preventing a mammal suffering from conditions caused by or contributed to by gram positive, gram negative or anaerobic bacteria comprising administering to a mammal in need thereof therapeutically effective amounts of one or more compounds or one or more pharmaceutical compositions disclosed herein.

Bacterial infection may be caused by one or more bacteria, for example, Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus or Enterobactericeae.

The conditions treated or prevented may be, for example, community-acquired pneumonia, upper and lower respiratory tract infections, skin and soft tissue infections, hospital-acquired lung infections or bone and joint infections, or other bacterial infections, for example, mastitis, catheter infection, foreign body, prosthesis infections or peptic ulcer disease.

In accordance with a third aspect, provided herein are processes for preparing the described compounds.

The term “alkyl,” unless otherwise specified, refers to a monoradical branched or unbranched saturated hydrocarbon chain having from 1 to 20 carbon atoms. Alkyl groups can be optionally interrupted by atom(s) or group(s) independently selected from oxygen, sulfur, a phenylene, sulphinyl, sulphonyl group or —NR_{b}—, wherein R_{b} can be hydrogen, alkyl, alkenyl, alkynyl cycloalkyl or aryl. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further (referred herein as “substituted alkyl”) with one or more substituents selected from alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, aryl, heterocyclyl, heteroaryl, (heterocyclyl)alkyl, cycloalkoxy, —CH═N—O(C₁₋₆alkyl), —CH═N—NH(C₁₋₄alkyl), —CH═N—NH(C₁₋₆alkyl)-C₁₋₆alkyl, arylthio, thiol, alkylthio, aryloxy, nitro, aminosulfonyl, aminocarbonylamino, —NHC(═O)Rₚ, —NRₚR_{q}, —C(═O)NRₚR_{q}, —NHC(═O)NRₚR_{q}, —C(═O)heteroaryl, C(═O)heterocyclyl, —O—C(═O)NRₚR_{q} {wherein Rₚ and R_{q} are independently selected from hydrogen, alkyl, alkenyl, cycloalkyl, alkoxy, cycloalkenyl, aryl, aralkyl, heterocyclyl, heteroaryl, heterocyclylalkyl, heteroarylalkyl}, nitro, hydroxyamino, alkoxyamino or S(O)ₘR₆₆ (wherein m is an integer from 0-2 and R₆₆ is alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, alkenyl, alkynyl, carboxy, —NRₚR_{q}, —C(═O)NRₚR_{q}, —OC(═O)NRₚR_{q}, —NHC(═O)NRₚR_{q} (wherein Rₚ and R_{q} are the same as defined earlier), hydroxy, alkoxy, halogen, CF₃, cyano, and S(O)ₘR₆₆ (wherein m is an integer from 0-2 and R₆₆ are the same as defined earlier); or an alkyl group also may be interrupted by 1-5 atoms of groups independently selected from oxygen, sulfur or —NR_{b}— {wherein R_{b} is selected from hydrogen, alkyl, cycloalkyl, alkenyl, cycloalkenyl, alkynyl, aryl, acyl, aralkyl, —C(═O)ORₚ (wherein Rₚ is the same as defined earlier), S(O)ₘR₆₆ (wherein m is an integer from 0-2 and R₆₆ is as defined earlier), or —C(═O)NRₚR_{q} (wherein Rₚ and R_{q} are as defined earlier)}. Unless otherwise constrained by the definition, all substituents may be substituted further by 1-3 substituents selected from alkyl, carboxy, carboxyalkyl, —NRₚR_{q}, —C(═O)NRₚR_{q}, —O—C(═O)NRₚR_{q} (wherein Rₚ and R_{q} are the same as defined earlier) hydroxy, alkoxy, halogen, CF₃, cyano, and S(O)ₘR₆₆ (wherein m is an integer from 0-2 and R₆₆ is same as defined earlier); or an alkyl group as defined above that has both substituents as defined above and is also interrupted by 1-5 atoms or groups as defined above.

As used herein the term “alkylene” refers a divalent group derived from a straight or branched chain saturated hydrocarbon having from 1 to 20 carbon atoms by the removal of two hydrogen atoms, for example, methylene, 1,2 ethylene and the like.

The term “alkenyl,” unless otherwise specified, refers to a monoradical of a branched or unbranched unsaturated hydrocarbon group having from 2 to 20 carbon atoms with cis, trans, or geminal geometry. It can be optionally interrupted by atom(s) or group(s) independently chosen from oxygen, sulfur, phenylene, sulphinyl, sulphonyl and —NR_{b}—, wherein R_{b} can be hydrogen, alkyl, alkenyl, alkynyl, cycloalkyl or aryl. In the event that alkenyl is attached to a heteroatom, the double bond cannot be alpha to the heteroatom. Alkenyl groups may be substituted further (referred to herein as “substituted alkenyl”) with one or more substituents selected from alkyl, alkenyl, alkynyl, alkoxy, cycloalkyl, cycloalkenyl, acyl, acylamino, acyloxy, —NHC(═O)Rₚ, —NRₚR_{q}, —C(═O)NRₚR_{q}, —NHC(═O)NRₚR_{q}, —O—C(═O)NRₚR_{q} (wherein Rₚ and R_{q} are the same as defined earlier), alkoxycarbonylamino, azido, cyano, halogen, hydroxy, oxo, keto, carboxyalkyl, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, hydroxyamino, alkoxyamino, nitro, or SO₂R₆₆ (wherein R₆₆ are is same as defined earlier). Unless otherwise constrained by the definition, alkenyl substituents optionally may be substituted further by 1-3 substituents selected from alkyl, carboxy, hydroxy, alkoxy, halogen, —CF₃, cyano, —NRₚR_{q}, —C(═O)NRₚR_{q}, —O—C(═O)NRₚR_{q} (wherein Rₚ and R_{q} are the same as defined earlier) and —SO₂R₆₆ (wherein R₆₆ is same as defined earlier). Groups, such as ethenyl or vinyl (CH═CH₂), 1-propylene or allyl (—CH₂CH═CH₂), iso-propylene (—C(CH₃)═CH₂), bicyclo[2.2.1]heptene, and the like, exemplify this term.

The term “alkynyl,” unless otherwise specified, refers to a monoradical of an unsaturated hydrocarbon, having from 2 to 20 carbon atoms. It can be optionally interrupted by atom(s) or group(s) independently chosen from oxygen, sulfur, phenylene, sulphinyl, sulphonyl and —NR_{b}—, wherein R_{b} can be hydrogen, alkyl, alkenyl, alkynyl, cycloalkyl or aryl. In the event that alkynyl is attached to a heteroatom, the triple bond cannot be alpha to the heteroatom. Alkynyl groups may be substituted further (referred to herein as “substituted alkynyl”) with one or more substituents selected from alkyl, alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, arylthio, thiol, alkylthio, aryl, aralkyl, aryloxy, aminosulfonyl, aminocarbonylamino, hydroxyamino, alkoxyamino, nitro, heterocyclyl, heteroaryl, heterocyclylalkyl, heteroarylalkyl, —NHC(═O)Rₚ, —NRₚR_{q}, —NHC(═O)NRₚR_{q}, —C(═O)NRₚR_{q}, —O—C(═O)NRₚR_{q} (wherein Rₚ and R_{q} are the same as defined earlier), S(O)ₘR₆₆ (wherein m is an integer from 0-2 and R₆₆ is as defined earlier). Unless otherwise constrained by the definition, alkynyl substituents optionally may be substituted further by 1-3 substituents selected from alkyl, carboxy, carboxyalkyl, hydroxy, alkoxy, halogen, CF₃, —NRₚR_{q}, —C(═O)NRₚR_{q}, —NHC(═O)NRₚR_{q}, —C(═O)NRₚR_{q} (wherein Rₚ and R_{q} are the same as defined earlier), cyano, or S(O)ₘR⁶⁶ (wherein m is an integer from 0-2 and R⁶⁶ is same as defined earlier). Groups such as ethynyl, (—C≡CH), propargyl (or propynyl, —CH₂C≡CH), and the like exemplify this term.

As used herein the term “alkenylene or alkynylene” refers to a divalent group derived from a straight or branched unsaturated hydrocarbon chain having from 2 to 20 carbon atoms by the removal of two hydrogen atoms, for example, vinylene, and the like.

As used herein the term “cycloalkyl” refers to saturated carbocyclic ring having three to seven carbon atoms. One or more hydrogen atom(s) of said cycloalkyl can be replaced by halogen, hydroxy, mercapto, alkoxy or thioalkyl. Examples of cycloalkyl include, but are not limited to, cyclopropyl, cyclobutyl and cyclopentyl, and the like.

As used herein the term “halogen or halo” refers to fluorine, chlorine, bromine or iodine.

“Amidoarylalkyl”, “Amidoarylalkenyl” refer to substituents wherein an aryl group is linked to the substituted moiety through an amido and an alkyl or alkenyl respectively.

The term “protecting group(s)” refers to moieties that prevent chemical reaction at a location of a molecule intended to be left unaffected during chemical modification of such molecule. Unless otherwise specified, protecting groups may be used on groups, such as hydrogen, hydroxy, amino, or carboxy. Examples of protecting groups are found in T. W. Greene and P. G. M. Wuts, “Protective Groups in Organic Synthesis”, 2 d Ed., John Wiley and Sons, New York, N.Y., which is incorporated herein by reference. The species of the carboxylic protecting groups, amino protecting groups or hydroxy protecting groups employed are not critical, as long as the derivatised moieties/moiety is/are stable to conditions of subsequent reactions and can be removed without disrupting the remainder of the molecule. For example, hydroxy protecting groups include, but are not limited to, trialkylsilyl, benzyloxycarbonyl, acid remainder, acyl, aroyl, alkyl, aryl, butyldiphenylsilyl, methoxymethyl and methylthiomethyl, and the like. Acid remainder can be acetic acid, propionic acid, maleic acid, tartaric acid, methane-sulfonic acid, benzene-sulphonic acid, p-toluenesulphonic acid, hydrochloric acid, hydrobromic acid, sulfuric acid, phosphoric acid, stearic acid, ethylsuccinic acid or laurylsulphonic acid.

As used herein the terms “thio” or “mercapto” refer to the group —SH.

As used herein the term “alkoxy” refers to a group O—R⁵ wherein R⁵ refers to alkyl, aryl or cycloalkyl as defined herein. Examples of alkoxy include, but are not limited to, methoxy, ethoxy, cyclopentoxy, phenoxy and the like.

As used herein the term “thioalkyl” refers to —SR⁵ wherein R⁵ is alkyl or cycloalkyl as defined herein.

As used herein the term “haloalkyl” refers to alkyl, as defined herein, of which one or more hydrogen(s) is/are replaced by halogen.

The term “aryl” herein refers to aromatic system having 6 to 14 carbon atoms, wherein the ring system can be mono-, bi- or tricyclic and are carbocyclic aromatic groups. For example, aryl groups include, but are not limited to, phenyl, biphenyl, anthryl or naphthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COORₛ (wherein Rₛ is hydrogen, alkyl, alkenyl, cycloalkyl, aralkyl, heterocyclylalkyl, heteroarylalkyl), NHC(═O)Rₚ, —NRₚR_{q}, —C(═O)NRₚR_{q}, —NHC(═O)NRₚR_{q}, —O—C(═O)NRₚR_{q}, S(O)ₘR₆₆ (wherein m is an integer from 0-2 and R₆₆ is same as defined earlier), carboxy, optionally substituted heterocyclyl, heteroaryl, heterocyclylalkyl, heteroarylalkyl, amino carbonyl amino, mercapto, haloalkyl, optionally substituted aryl, optionally substituted heterocyclylalkyl, thioalkyl, —CONHRₚ, —OCORₚ, —CORₚ, —NHSO₂Rₚ, or —SO₂NHRₚ (wherein Rₚ and R_{q} are the same as defined earlier). The aryl group optionally may be fused with a cycloalkyl group, wherein the cycloalkyl group may optionally contain heteroatoms selected from O, N or S. Groups such as phenyl, naphthyl, anthryl, biphenyl, and the like exemplify this term.

As used herein the term “aralkyl” stands for an aryl radical having 7 to 14 carbon atoms, which is bonded to an alkylene chain, as defined herein. Examples of aralkyl include, but are not limited to, benzyl, napthylmethyl, phenethyl and phenylpropyl, and the like.

The term “heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted,

wherein the substituents are selected from halogen (e.g., F, Cl, Br, I), hydroxy, alkyl, alkenyl, alkynyl, cycloalkyl, acyl, optionally substituted aryl, alkoxy, alkaryl, cyano, nitro, oxo, carboxy, optionally substituted heterocyclyl, optionally substituted heterocyclylalkyl, optionally substituted heteroaryl, —O—C(═O)Rₚ, —O—C(═O)ORₚ, —C(═O)NRₚR_{q}, S(O)ₘR₆₆, —O—C(═O)NRₚR_{q}, —NHC(═O)NRₚR_{q}, —NRₚR_{q}, NRₚR_{q}, mercapto, haloalkyl, thioalkyl, —COORₚ, —COONHRₚ, —CORₚ, —NHSO₂Rₚ, SO₂NH Rₚ (wherein m, R₆₆, Rₚ and R_{q} are as defined earlier) or guanidine. Such ring systems can be mono-, bi- or tricyclic. Carbonyl or sulfonyl group can replace carbon atom(s) of heterocyclyl. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, benzoxazinyl, benzthiazinyl, imidazolyl, benzimidazolyl, tetrazolyl, carbaxolyl, indolyl, phenoxazinyl, phenothiazinyl, dihydropyridinyl, dihydroisoxazolyl, dihydrobenzofuryl, azabicyclohexyl, thiazolidinyl, dihydroindolyl, pyridinyl, isoindole 1,3-dione, piperidinyl, tetrahydropyranyl, piperazinyl, 3H-imidazo[4,5-b]pyridine, isoquinolinyl, 1H-pyrrolo[2,3-b]pyridine, 4-pyridyl-3-yl-imidazol-1-yl, 4-phenyl-imidazol-1-yl, 4-thiophen-3-yl-imidazol-1-yl, and the like.

As used herein the term “(heterocyclyl)alkyl” stands for heterocyclyl which is bonded to an alkylene chain. Examples of heterocyclyl alkyl include, but are not limited to, isothiazolidinyl ethyl, isothiazolyl propyl, pyrazinyl methyl, pyrazolinyl propyl and pyridyl butyl, pyridyl methyl and the like.

As used herein the term “polymorphs” includes all crystalline form and amorphous form for compounds described herein. In addition, some of the compounds described herein may form solvates with water (i.e., hydrate, hemihydrate or sesquihydrate) or common organic solvents. Such solvates are also encompassed within the scope of this invention.

As used herein the term “prodrugs” refers to the compounds that are rapidly transformed in vivo to yield the parent compound of the previous formula.

The phrase “pharmaceutically acceptable salts” denotes salts of the free base, which possess the desired pharmacological activity of the free base and which are neither biologically nor otherwise undesirable. Suitable pharmaceutically acceptable salts may be prepared from an inorganic or organic acid. Example of such inorganic acids include, but not limited to, hydrochloric, hydrobromic, hydroiodic, carbonic, sulfuric, phosphoric acid and like. Appropriate organic acids include, but not limited to, aliphatic, cycloaliphatic, aromatic, heterocyclic, carboxylic and sulfonic classes of organic acids, for example, formic, acetic, propionic, succinic, glycolic, gluconic, lactic, malic, tartaric, citric, ascorbic, glucuronic, maleic, fumeric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, beta-hydroxybutyric, cyclohexylaminosulfonic, galactaric and galactironic acid and the like.

The term “pharmaceutically acceptable carriers” is intended to include non-toxic, inert solid, semi-solid or liquid filler, diluent, encapsulating material or formulation auxiliary of any type.

The compounds of present invention include stereoisomers. The term “stereoisomer” refers to compounds, which have identical chemical composition, but differ with regard to arrangement of the atoms and the groups in space. These include enantiomers, diastereomers, geometrical isomers, atropisomer and comformational isomers. Geometric isomers may occur when a compound contains a double bond or some other feature that gives the molecule a certain amount of structural rigidity. An enantiomer is a stereoisomer of a reference molecule that is the nonsuperimposable mirror image of the reference molecule. A diastereomer is a stercoisomer of a reference molecule that has a shape that is not the mirror image of the reference molecule. An atropisomer is a conformational of a reference compound that converts to the reference compound only slowly on the NMR or laboratory time scale. Conformation isomers (or conformers or rotational isomers or rotamers) are stereoisomers produced by rotation about a bonds, and are often rapidly interconverting at room temperature. Racemic mixtures are also encompassed within the scope of this invention.

The term “drug resistance” or “drug resistant” refers to the characteristics of a microbe to survive in presence of a currently available antimicrobial agent such as an antibiotic at its effective concentration.

The term “subject” includes any animal or artificially modified animal. As a particular embodiment, the subject is a human.

The term “administering” includes the treatment of the various disorders described with the compounds specifically disclosed or with a compound which may not be specifically disclosed, but which converts to the specified compound in vivo after administration to the patient.

The term “treating” means reversing, alleviating, inhibiting the progress of or preventing the disorder or condition to which such term applies, or one or more symptoms of such disorder or condition. The term “treatment” refers to the act of treating, as treating is defined above.

The compounds described herein may be prepared by techniques known to one of ordinary skill in the art. In addition, the compounds described herein may be prepared by the following reaction sequences as depicted in Schemes I, IA, II and III.

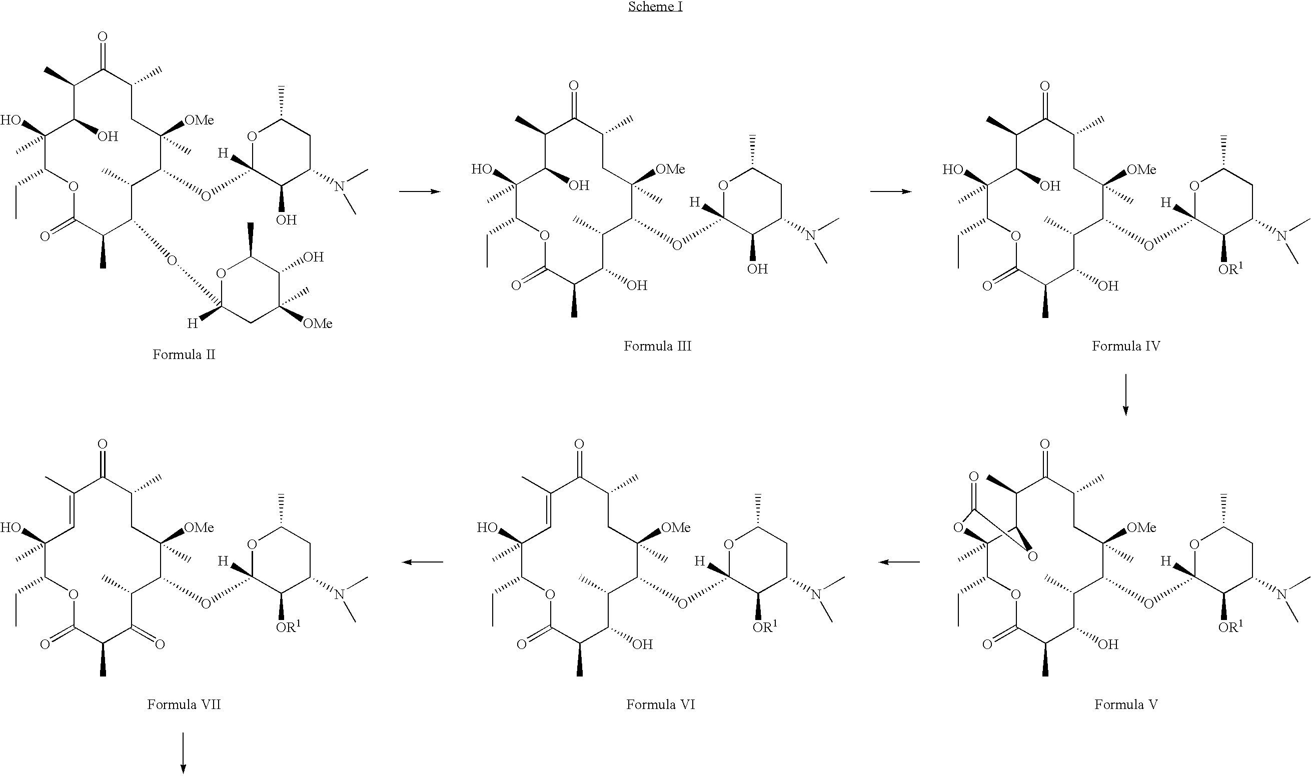

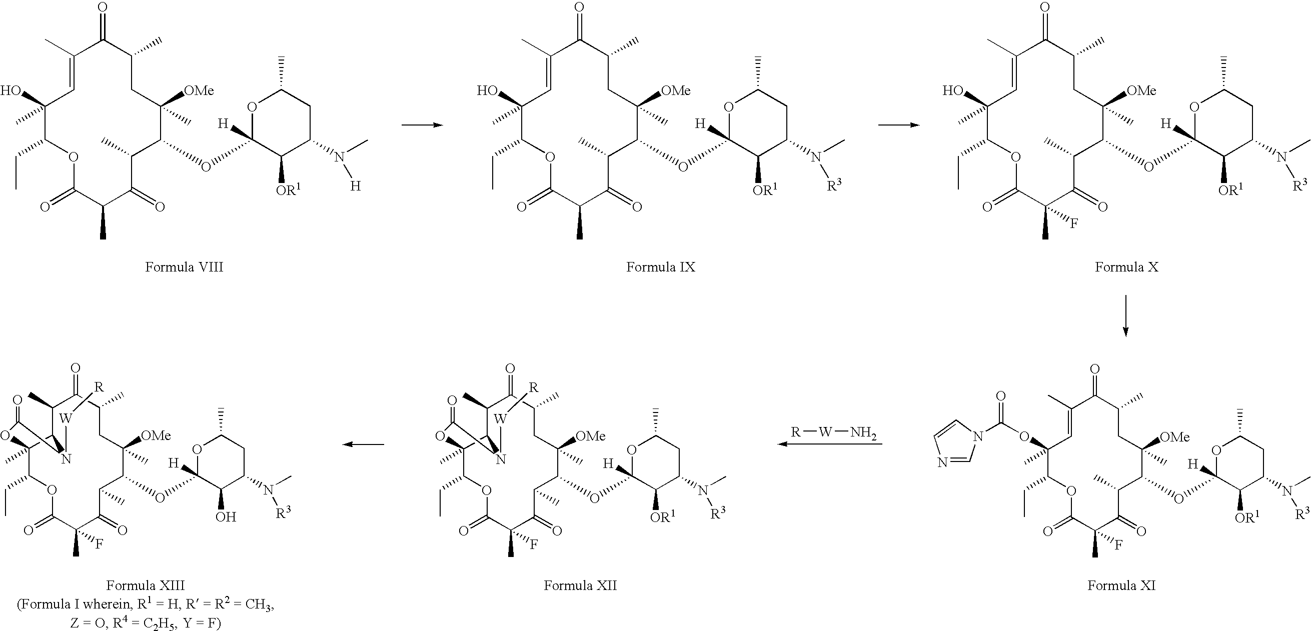

Compounds of Formula XIII can be prepared according to Scheme I. Thus, clarithromycin of Formula II can be hydrolyzed to form compounds of Formula III. Compounds of Formula III can be protected by reacting with one or more reagents of Formula R¹₂O or R¹X (wherein X is halogen and R¹ is as defined earlier) to form compounds of Formula IV. Compounds of Formula IV can be reacted with one or more reagents, for example, triphosgene, ethylene dicarbonate or a mixture thereof, to form compounds of Formula V. Compounds of Formula V can be reacted with one or more organic bases (for example, tetramethyl guanidine, trimethylamine or mixtures thereof) to form compounds of Formula VI. Compounds of Formula VI can be oxidized to form compounds of Formula VII. Compounds of Formula VII can be desmethylated at the 3′-N-dimethyl group to form compounds of Formula VIII. Compounds of Formula VIII can be alkylated with one or more reagents of Formula R³CHO, R³₂ CO or R³X (wherein X can be halogen) to form compounds of Formula IX (wherein R³ is the same as defined earlier). Compounds of Formula IX can be fluorinated to form compounds of Formula X. Compounds of Formula X can be reacted with N,N′-carbonyldiimidazole to form compounds of Formula XI. Compounds of Formula XI can be reacted with compounds of Formula R-W-NH₂ to form compounds of Formula XII (wherein W and R are the same as defined earlier). Compounds of Formula XII can be deprotected to form compounds of Formula XIII.

Clarithromycin of Formula II can be hydrolyzed in the presence of one or more inorganic or organic acids. Suitable inorganic or organic acids include, for example, hydrochloric acid, sulfuric acid, dichloroacetic acid or mixtures thereof.

Compounds of Formula III can be protected with one or more reagents of Formula R¹₂O or R¹X in one or more solvents, for example, dichloromethane, dichloroethane, chloroform, carbon tetrachloride, ethyl acetate or mixtures thereof. The protection reactions can also be carried out in the presence of one or more organic bases, for example, triethylamine, pyridine, tributylamine, diisopropylethylamine, 4-(N-dimethylamino)pyridine or mixtures thereof.

Compounds of Formula IV can be reacted with one or more reagents, for example, phosgene, diphosgene, triphosgene, ethylene carbonate or mixtures thereof. Compounds of Formula IV can also be reacted in one or more solvents, for example, dichloromethane, chloroform, carbon tetrachloride, dichloroethane or mixtures thereof. These reactions can also be carried out in the presence of one or more organic bases, for example, triethylamine, pyridine, tributylamine, 4-(N-dimethylamino) pyridine, diisopropylethylamine or mixtures thereof.

Compounds of Formula V can be reacted with one or more organic bases, for example, tetramethyl guanidine, trimethylamine or mixtures thereof. These reactions can also be carried out in one or more solvents, for example, dimethylformamide, tetrahydrofuran, dimethylsuiphoxide or mixtures thereof.

Compounds of Formula VI can be oxidized with one or more oxidizing agents, for example, Dess-Martin periodinane, N-chlorosuccinimide, pyridinium chlorochromate, Swern Oxidation reagent (oxalyl chloride and dimethylsulfoxide), Pfitzner-Moffatt Oxidation reagent (dicyclohexylcarbodiimide and dimethylsulfoxide), pyridinium dichromate, 1-ethyl-3-(3-dimethyl aminopropyl) carbodiimide hydrochloride or mixtures thereof. Compounds of Formula VI can also be oxidized in one or more solvents, for example, chloroform, dichloromethane, carbon tetrachloride, dimethylsulfoxide dichloroethane or mixtures thereof.

Compounds of Formula VII can be desmethylated in the presence of one or more desmethylating agents, for example, iodine in acetic acid, N-iodosuccinimide, 1-chloroethyl chloroformate, diisopropylazodicarboxylate or mixtures thereof. The desmethylation reactions can also be carried out in one or more solvents, for example, acetonitrile, tetrahydrofuran, dichloromethane, dichloroethane, chloroform, carbon tetrachloride, ethyl acetate or mixtures thereof. Such desmethylation reactions can be quenched in the presence of one or more quenching agents, for example, sodium bisulphite, sodium carbonate, cesium carbonate, potassium carbonate, sodium acetate or mixtures thereof.

Compounds of Formula VIII can be alkylated with one or more reagents of Formula R³CHO, R₂³CO or R³X. The alkylation reactions can also be carried out in one or more solvents, for example, dimethylformamide, acetonitrile, methanol, acetone, tetrahydrofuran or mixture thereof. The alkylation reactions can also be carried out in the presence of one or more inorganic or organic bases, for example, sodium hydrogen carbonate, potassium carbonate, sodium hydride, pyridine, triethylamine, sodium carbonate, sodium acetate, sodium thiosulphate, diisopropylethylamine or mixtures thereof.

Compounds of Formula IX can be fluorinated in presence of one or more fluorinating agents, for example, select fluor, N-fluorobenzene sulfonamide or mixtures thereof. The fluorination reactions can also be carried out by procedures disclosed by G. Sankar Lal and Syvret R. G., in Chem. Rev., 96, 1737-1755 (1996) (for example, dicarbonyl compounds (e.g., compounds of Formula IX) can be fluorinated with N-fluoropyridinium salts in the presence of a Lewis acid; or dicarbonyl compounds (e.g., compounds of Formula IX) can be fluorinated with Selectfluor in neutral conditions or by metal enolates). The fluorination reactions can also be carried out in one or more solvents, for example, dimethylformamide, tetrahydrofuran, dimethylsulphoxide or mixtures thereof. Further, the fluorination reactions can also be carried out in the presence of one or more inorganic bases, for example, potassium carbonate, sodium hydride, sodium acetate, sodium thiosulphate, potassium-t-butoxide, sodium-t-butoxide, lithium diisopropylamide, sodium methoxide, sodium ethoxide, potassium methoxide, potassium ethoxide, sodium isopropoxide, potassium isopropoxide, lithium carbonate or mixtures thereof.

Compounds of Formula X can be reacted with N,N′-carbonyldiimidazole in one or more solvents, for example, dimethylformamide, acetonitrile, tetrahydrofuran or mixture thereof. These reactions can also be carried out in the presence of one or more inorganic bases, for example, sodium hydrogen carbonate, sodium carbonate, potassium carbonate, sodium acetate, potassium-t-butoxide, sodium thiosulphate, sodium hydride or mixtures thereof.

Compounds of Formula XI can be reacted with compounds of Formula R-W-NH₂ in one or more solvent systems, for example, dimethylformamide, acetonitrile/water, dimethylformamide/water or combinations thereof.

Compounds of Formula XII can be deprotected in one or more alcohols, for example, methanol, ethanol, propanol, isopropanol or mixtures thereof.

Examples of compounds include (also shown in Table I):
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(3-pyridin-3-yl-phenoxy)-propyl)-imino)]erythromycin A (Compound No. 2),
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(3-pyridin-3-yl-phenoxy)-propyl)-imino)]erythromycin A (Compound No. 3),
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(pyridin-3-yloxy)-propyl)-imino)]erythromycin A (Compound No. 4),
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(3-thiophen-3-yl-phenoxy)-propyl)-imino)]erythromycin A (Compound No. 5),
   pharmaceutically acceptable salts, pharmaceutically acceptable solvates, stereoisomers, prodrugs, metabolites and polymorphs thereof

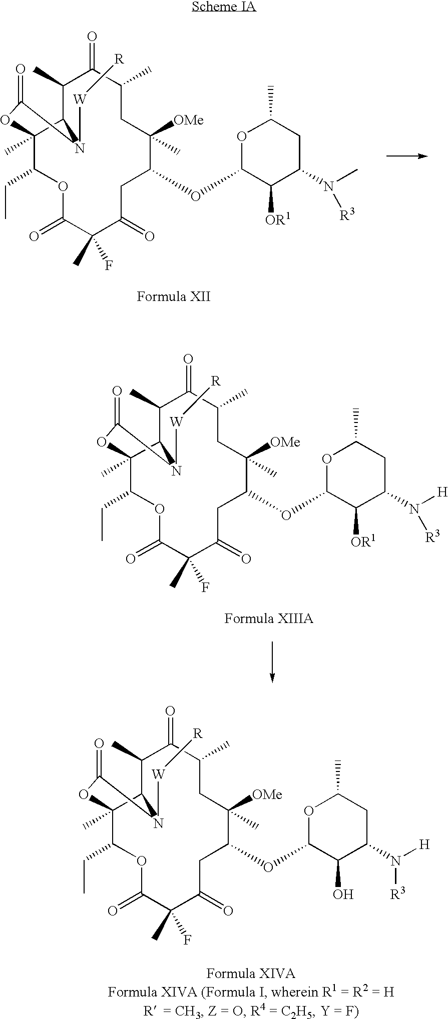

Compounds of Formula XIVA can be prepared according to Scheme IA. Thus, compounds of Formula XII can be desmethylated at the 3′-N-dimethyl group to form compounds of Formula XIIIA. Compounds of Formula XIIIA can be deprotected to form compounds of Formula XIVA.

Compounds of Formula XII can be desmethylated in one or more solvents, for example, acetonitrile, tetrahydrofuran, dichloromethane, dichloroethane, chloroform, carbon tetrachloride, ethyl acetate or mixture thereof. Desmethylation reactions can be quenched in the presence of one or more quenching agents, for example, sodium bisulphite, sodium carbonate, potassium carbonate, cesium carbonate, sodium acetate or mixture thereof.

Compounds of Formula XIIIA can be deprotected in one or more alcohols, for example, methanol, ethanol, propanol, isopropanol or mixtures thereof.

Examples of compounds include (also shown in Table I):
2-α-Fluoro-5-O-(3′-N-didesmethyl-3′-N-ethyl)-11,12-dideoxy-3-O-decladinosyl-6-O-methyl-3-oxo-12,11-[oxycarbonyl-((4-imidazol[4,5-b]pyridin-3-yl)-butyl)-imino]erythromycin A (Compound No. 1),
   pharmaceutically acceptable salts, pharmaceutically acceptable solvates, stereoisomers, prodrugs, metabolites and polymorphs thereof.

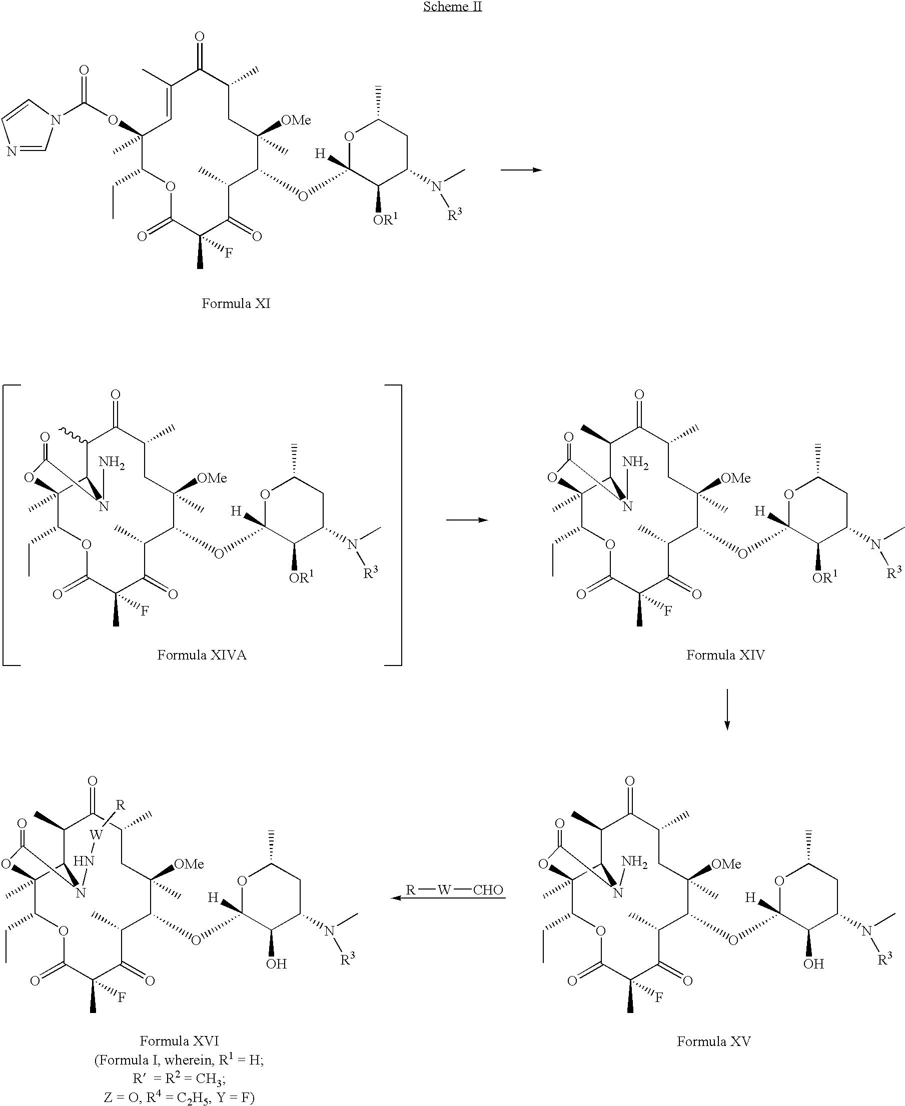

Compounds of Formula XVI can be prepared according to Scheme II. Thus, compounds of Formula XI can be reacted with hydrazine hydrate to form unsubstituted carbazate derivatives of Formula XIVA. The compounds of Formula XIVA on epimerization forms desired isomer of Formula XIV. Compounds of Formula XIV can be deprotected to form compounds of Formula XV. Compounds of Formula XV can be reacted with compounds of Formula R-W-CHO to form compounds of Formula XVI (wherein W and R are the same as defined earlier).

Compounds of Formula XI can be reacted with hydrazine hydrate to form carbazate epimers of Formula XIVA in one or more solvents, for example, dimethylformamide, acetonitrile, tetrahydrofuran, ethanol, methanol or mixtures thereof.

The epimers of Formula XIVA can be treated with one or more bases, for example, potassium-t-butoxide, sodium-t-butoxide or mixtures thereof, to form a desired isomer of Formula XIV. The reaction of epimers with one or more bases can also be carried out in one or more solvents, for example, tetrahydrofuran, dimethylformamide, acetonitrile, ethanol, methanol or mixtures thereof.

Compounds of Formula XIV can be reacted with compounds of Formula R-W-CHO in one or more alcohols, for example, methanol, ethanol, propanol, isopropanol, t-butyl alcohol or mixtures thereof. These reactions can also be carried out in presence of one or more reducing agents, for example, sodium cyanoborohydride, sodium borohydride, sodium triacetoxyborohydride or mixtures thereof. Further, these reactions can be carried out in presence of one or more organic acids, for example, acetic acid, formic acid, trifluoroacetic acid or mixtures thereof.

Compounds of Formula XV can be deprotected in one or more alcohols, for example, methanol, ethanol, propanol, isopropanol or mixtures thereof.

In the schemes herein, where specific bases, acids, solvents etc. are mentioned, it is understood that other bases, acids, solvents etc., known to one of ordinary skill in the art may also be use. Similarly, the reaction temperature and duration of the reactions may be adjusted accordingly.

Examples of compounds include (also shown in Table I)
2-α-Fluoro-5-O-(3′-N-desmethyl-3′-N-ethyl)-11,12-dideoxy-3-O-decladinosyl-6-O-methyl-3-oxo-12,11-[oxycarbonyl-hydrazo]erythromycin A (Compound No. 6),
2-α-Fluoro-5-O-(3′-N-desmethyl-3′-N-allyl)-11,12-dideoxy-3-O-decladinosyl-6-O-methyl-3-oxo-12,11-[oxycarbonyl-hydrazo]erythromycin A (Compound No. 7),
2-α-Fluoro-5-O-(3′-N-desmethyl-3′-N-allyl)-11,12-dideoxy-3-O-decladinosyl-6-O-methyl-3-oxo-12,11-[oxycarbonyl-((3-pyrrolo[2,3-b]pyridin-1-yl)-propyl)-hydrazo)]erythromycin A (Compound No. 8),
2-α-Fluoro-5-O-(3′-N-desmethyl-3′-N-allyl)-11,12-dideoxy-3-O-decladinosyl-6-O-methyl-3-oxo-12,11-[oxycarbonyl-((3-phenylpropyl)-hydrazo)]erythromycin A (Compound No. 9),
2-α-Fluoro-5-O-(3′-N-desmethyl-3′-N-allyl)-11,12-dideoxy-3-O-decladinosyl-6-O-methyl-3-oxo-12,11-[oxycarbonyl-((3-isoquinolin-5-yl-propyl)-hydrazo)]erythromycin A (Compound No. 10),
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-phenylpropyl)-hydrazo)]erythromycin A (Compound No. 11),
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-phenyl-imidazol-1-yl)-propyl)-hydrazo)]erythromycin A (Compound No. 12),
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-phenyl-imidazol-1-yl)-propyl)-hydrazo)]erythromycin A (Compound No. 13),
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-benzoimidazol-1-yl)-propyl)-hydrazo)]erythromycin A (Compound No. 14),
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-pyridyl-3-yl-imidazol-1-yl)-propyl)-hydrazo)]erythromycin A (Compound No. 15),
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-pyridyl-3-yl-imidazol-1-yl)-propyl)-hydrazo)]erythromycin A (Compound No. 16),
2-α-Fluoro-5-O-3′-N-desmethyl-3′-N-allyl)-11,12-dideoxy-3-O-decladinosyl-6-O-methyl-3-oxo-12,11-[oxycarbonyl-((3-benzoimidazol-1-yl)-propyl)-hydrazo)]erythromycin A (Compound No. 17),
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-thiophen-3-yl-imidazol-1-yl)propyl)-hydrazo)]erythromycin A (Compound No. 18),
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-pyridyl-3-yl-imidazol-1-yl)-butyl)-hydrazo)]erythromycin A (Compound No. 22),
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-phenyl-imidazol-1-yl)-butyl)-hydrazo)]erythromycin A (Compound No. 23),
2α-Fluoro-5-O-(3′-N-desmethyl-3′-N-ethyl)-11,12-dideoxy-3-O-decladinosyl-6-O-methyl-3-oxo-12,11-[oxycarbonyl-((3-phenyl-allyl)-hydrazo)]erythromycin A (Compound No. 24),
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-(2-chloro-pyrimidin-5-yl)-imidazol-1-yl)-propyl)-hydrazo)]erythromycin A (Compound No. 25),
   pharmaceutically acceptable salts, pharmaceutically acceptable solvates, stereoisomers, prodrugs, metabolites and polymorphs thereof.

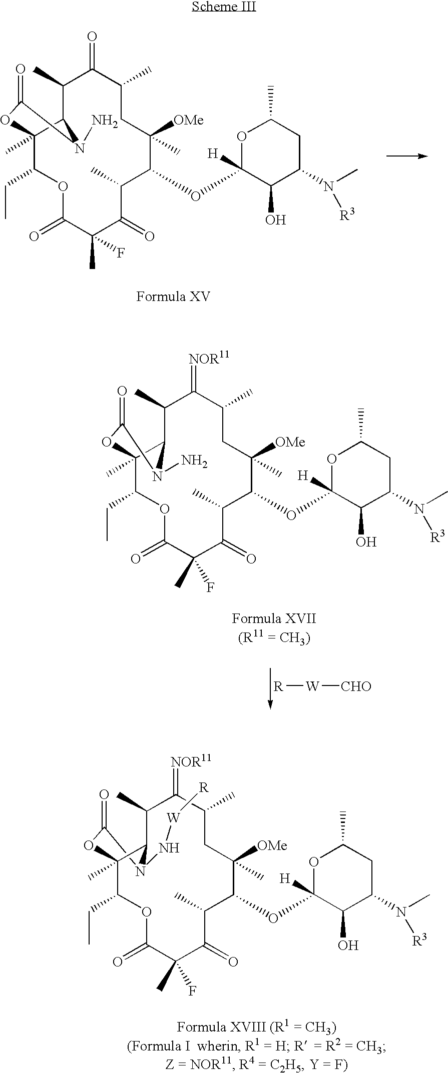

Compounds of Formula XVIII can be prepared according to Scheme III. Thus, compounds of Formula XV can be treated with one or more reagents of Formula H₂NOR¹¹ (wherein R¹¹ is the same as defined earlier) to form compounds of Formula XVII. Compounds of Formula XVII can be reacted with compounds of Formula R-W-CHO (wherein W and R are the same as defined earlier) to form compounds of Formula XVIII.

Compounds of Formula XV can be reacted with compounds of Formula H₂NOR¹¹ in one or more solvents, for example ethanol, methanol, isopropanol or mixtures thereof.

Compounds of Formula XVH can be reacted with compounds of Formula R-W-CHO in one or more solvents, for example, ethanol, methanol, isopropanol, tetrahydrofuran dimethylformamide or mixtures thereof. These reactions can also be carried out in presence of one or more reducing agents, for example, sodium cyanoborohydride, sodium borohydride sodium triacetoxyborohydride or mixtures thereof. Further, these reactions can also be carried out in presence of one or more organic acids, for example, acetic acid, formic acid trifluoroacetic acid or mixtures thereof.

Examples of compounds include (also shown in Table I):
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-phenyl-imidazol-1-yl)-propyl)-hydrazo)]erythromycin A-9-(O-methyl)oxime (Compound No. 19),
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-pyridyl-3-yl-imidazol-1-yl)-propyl)-hydrazo)]erythromycin A-9-(O-methyl)oxime (Compound No. 20),
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-thiophen-3-yl-imidazol-1-yl)propyl)-hydrazo)]erythromycin A-9-(O-methyl)oxime (Compound No. 21),
   pharmaceutically acceptable salts, pharmaceutically acceptable solvates, stereoisomers, prodrugs, metabolites and polymorphs thereof.

In the above schemes, where specific bases, acids, solvents etc. are mentioned, it is to be understood that other bases, acids, solvents etc., known to those skilled in the art may also be use. Similarly, the reaction temperature and duration of the reactions may be adjusted according to the desired needs. All the epimers, unless otherwise specified in the above schemes, are also encompassed within the scope of this invention.

TABLE I
Non-limiting examples of compounds of Formula I

| | |
|---|---|
| | Fomula I |
| 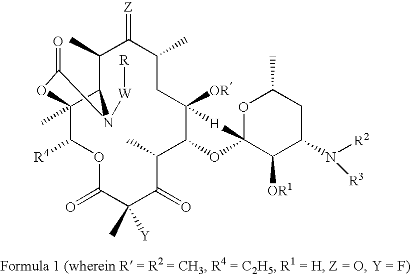 | |

| Compound | | | |
|---|---|---|---|
| No. | W | R | R³ |
| 1* | —(CH₂)₄— | 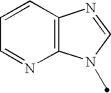 | —C₂H₅ |
| 2 | —(CH₂)₃O— | 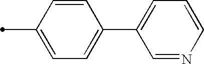 | —C₂H₅ |
| 3 | —(CH₂)₃O— | 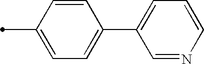 | —CH₂CH═CH₂ |
| 4 | —(CH₂)₃O— | 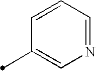 | —C₂H₅ |
| 5 | —(CH₂)₃—O— | 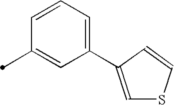 | —C₂H₅ |
| 6 | —NH— | H | —C₂H₅ |
| 7 | —NH— | H | —CH₂CH═CH₂ |
| 8 | —NH(CH₂)₃— | 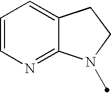 | —CH₂CH═CH₂ |
| 9 | —NH(CH₂)₃— | 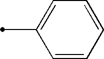 | —CH₂CH═CH₂ |
| 10 | —NH(CH₂)₃— | 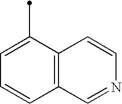 | —CH₂CH═CH₂ |
| 11 | —NH(CH₂)₃— | 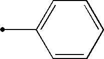 | —C₂H₅ |
| 12 | —NH(CH₂)₃— | 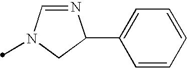 | —CH₂CH═CH₂ |
| 13 | —NH(CH₂)_{3—} | 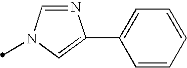 | —C₂H₅ |
| 14 | —NH(CH₂)₃— | 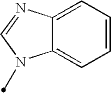 | —C₂H₅ |
| 15 | —NH(CH₂)₃— | 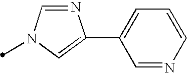 | —C₂H₅ |
| 16 | —NH(CH₂)₃— | 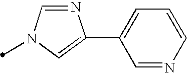 | —CH₂CH═CH₂ |
| 17 | —NH(CH₂)₃— | 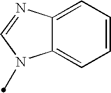 | —CH₂CH═CH₂ |
| 18 | —NH(CH₂)_{—} | 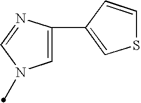 | —CH₂CH═CH₂ |
| 19** | —NH(CH₂)₃— | 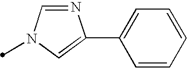 | —CH₂CH═CH₂ |
| 20** | —NH(CH₂)₃— | 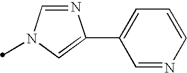 | —C₂H₅ |
| 21** | —NH(CH₂)₃— | 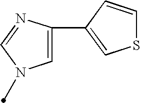 | —CH₂CH═CH₂ |
| 22 | —NH—(CH₂)₂CH(CH₃)— | 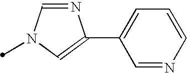 | —CH₂CH═CH₂ |
| 23 | —NH—(CH₂)₂CH(CH₃)— | 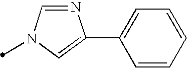 | —CH₂CH═CH₂ |
| 24 | —NH—CH₂—CH═CH— | 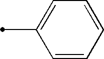 | —C₂H₅ |
| 25 | —NH—(CH₂)₃— | 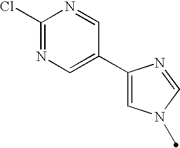 | —C₂H₅ |

*R² is Hydrogen,
**Z is NOMe, [symbol]
is point of attachment

Because of their antibacterial activity, compounds described herein may be administered to an animal for treatment orally, topically, rectally, internasally, intracistemally, intravaginally, intraperitoneally, buccally or by parenteral route. The pharmaceutical compositions of the present invention comprise a pharmaceutically effective amount of compounds described herein formulated together with one or more pharmaceutically acceptable carriers.

The compounds described herein can also be administered concurrently or sequentially in any order with one or more additional therapeutic agents. For example, one or more additional therapeutic agents include, agents useful in treating propionibacterium acnes and any other gram positive bacteria associated with acne vulgaris, endocarditis, anaerobic arthritis, wound infections and abscesses. The one or more additional therapeutic agents include, for example, benzoyl peroxide, clindamycin, telithromycin, tretinoin, vitamin E, vitamin A and its derivatives, tetracycline, isotretinoin, vitamin C, vitamin D, chaparral, dandelion root, licoric root, Echinacea, kelp, cayenine, sassafras, elder flowers, pantothenic acid, para amino benzoic acid, biotin, cholin, inositol, folic acid, calcium, magnesium, potassium, vitamin B₆, zinc, carotenoid orazelaic acid or mixtures thereof.

Pharmaceutical compositions for use in the methods described herein may be prepared by any of the methods of pharmacy, but all methods include the step of bringing into association the active ingredient(s) with the carrier which constitutes one or more necessary ingredients. In general, the compositions are prepared by uniformly and intimately admixing the active ingredient with pharmaceutically acceptable liquid carriers or finely divided solid carriers or both, and then, if necessary, shaping the product into the desired presentation. The active ingredients formulated in such pharmaceutical compositions can include one or more compounds described herein, and optionally one or more additional therapeutic agents described above.

Solid form preparations include powders, tablets, pills, dispersible granules, dragees, capsules, cachets, suppositories, troches, patches, gel caps, magmas, lozenges, creams, pastes, plasters, lotions, discs, or ointments. Liquid form preparations include solutions, suspensions, emulsions, microemulsions, syrups, elixirs, aerosols, nasal spays or oral sprays.

Solid carriers can include one or more substances, which may also act as diluents, flavoring agents, solubilizers, lubricants, suspending agents, binders, or disintegrating agents. Solid carriers can also include finely divided solids, which can be in admixture with one or more finely divided compounds described herein.

In preparing tablets, one or more compounds described herein can be mixed with one or more carriers having the necessary binding properties in suitable proportions and compacted into the desired shape and size. In some embodiments, powders and tablets can contain from about 5 to about 70 percent of one or more compounds described herein. Suitable solid carriers include, for example, sucrose, glucose, lactose, pectin, mannitol, silicic acid, dextrin, starch, gelatin, tragacanth, low melting wax, cocoa butter sugars, sodium citrate, dicalcium phosphate, microcrystalline cellulose, granulating agents, lubricants, binders, disintegrating agents, absorption accelerators, wetting agents, adsorbents and the like. Binders include, for example, carboxymethylcellulose, alginates, gelatins, polyvinylpyrrolidinone, sucrose, acacia; disintegrating agents include, for example, agar-agar, calcium carbonate, potato starch, alginic acid, certain silicates and sodium carbonate; absorption accelerators include, for example, quaternary ammonium compounds; wetting agents include, for example, cetyl alcohol, glycerol mono stearate; adsorbents include, for example, Kaolin; lubricants include, for example, talc, calcium stearate, magnesium stearate, solid polyethyleneglycol, sodium lauryl sulphate and mixture thereof. In the case of capsules, tablets, pills, the dosage form may also comprise buffering agents. For example, a tablet may be prepared by compression or molding, optionally, with one or more accessory ingredients. Compressed tablets may be prepared by compressing in a suitable machine the active ingredient in a free-flowing form such as powder or granules, optionally mixed with one or more binders, lubricants, inert diluents, surface active or dispersing agents. Molded tablets may be made by molding, in a suitable machine, a mixture of a powdered form of one or more compounds moistened with one or more inert liquid diluents.

For liquid form preparations, active compounds can be mixed with water or other solvent, solubilizing agents and emulsifiers, for example, ethyl alcohol, isopropanol, ethyl carbonate, ethyl acetate, benzyl alcohol, benzyl benzoate, propylene glycol, 1,3-butylene glycol, dimethylformamide, oils (for example, cottonseed, groundnut, corn, germ, olive, castor and sesame oil), glycerol, fatty acid esters of sorbitan or mixtures thereof.

Aqueous solutions suitable for oral use can be prepared by dissolving the active component in water and adding suitable colorants, flavors, stabilizers, wetting agents, emulsifying agents, suspending agents, sweetening agents, flavoring agents, perfuming agents and thickening agents as desired. Aqueous suspension suitable for oral use can be made by dispersing the finely divided active component in water with viscous material, for example, natural or synthetic gums, resins, methyl cellulose, sodium carboxymethyl cellulose and other suspending agents. Other liquid form preparations include, for example, water or water-propylene glycol solutions for parenteral injection. Other injectable preparations, for example, sterile injections, injectable depot forms, aqueous suspensions may be formulated according to the art using suitable dispersing or wetting and suspending agent. Among the acceptable vehicles and solvents that may be employed are water, Ringer's solution and isotonic sodium chloride.

Such solutions are prepared so as to be acceptable to biological systems with respect to isotonicity, pH, and other parameters. Liquid preparations can also be formulated in solution in aqueous polyethylene glycol solution.

Ointment preparations can contain one or more compounds described herein or salts thereof with a physiologically acceptable carrier. Such salts can be heavy metal salts. The carrier can desirably be a conventional water-dispersible hydrophilic or oil-in-water carrier, particularly a conventional semi-soft or cream-like water-dispersible or water soluble, oil-in-water emulsion infected surface with a minimum of discomfort. Suitable compositions may be prepared by merely incorporating or homogeneously admixing finely divided compounds with the hydrophilic carrier or base or ointment.

Dosage forms for tropical or transdermal administration of one or more compounds described herein includes ointments, pastes, creams, lotions, gels, powders, solutions, sprays, inhalants or patches. Active compounds can be admixed under sterile condition with one or more pharmaceutically acceptable carriers and any desired preservatives or buffers as may be required. Ophthalmic formulations, eardrops, eye ointments, powders and solutions are also encompassed within the scope of this invention.

The pharmaceutical preparation can be in unit dosage form. In such forms, the preparation can be subdivided into unit doses containing appropriate quantities of the active component, i.e., one or more compounds described herein and optionally one or more other therapeutic agents. Dosage forms can be a packaged preparation containing one or more discrete unit dosages, for example, capsules; tablets; powders in vials, capsules or ampoules; ointments; cachets; gels or gel caps; cream itself; dispersible granules; suppositories; troches; patches; magmas; lozenges; pastes; plasters; lotions; discs; ointments; solutions; suspensions, emulsions, syrups, elixirs, aerosols, nasal spays or oral sprays.

The magnitude of a prophylactic or therapeutic dose of one or more compounds described herein in the acute or chronic prevention, treatment, or management of a disorder or condition will vary with the severity of the condition to be treated and the route of administration. The dose, and perhaps the dose frequency, will also vary according to the age, body weight, and response of the individual patient. Suitable total daily dose ranges can be readily determined by those skilled in the art. In general, the total daily dose range for one or more compounds described herein, for the conditions described herein, is from about 1 mg to about several grams administered in single or divided doses according to the particular application and the potency of the active ingredient. Compounds described herein can also be administered at initial dosages of about 3 mg to about 40 mg per kilogram daily, preferably from about 5 mg to about 25 mg per kilogram daily, more preferably from about 10 g to about 15 mg per kilogram daily. Suitable dosage amounts can be determined using small dosages that are less than the optimum dose. Such small dosages can be increased in small increments until the optimum effect is reached. Dosage amounts may be divided and administered as divided doses if desired.

Any suitable route of administration may be employed for providing the patient with an effective dosage of one or more compounds described herein according to the methods of the present invention. For example, oral, intraoral, rectal, parenteral, epicutaneous, transdermal, subcutaneous, intramuscular, intranasal, sublingual, buccal, intradural, intraocular, intrarespiratory, or nasal inhalation and like forms of administration may be employed. Oral administration is generally preferred.

In addition to the common dosage forms set out above, the compound for use in the methods of the present invention may also be administered by controlled release means and/or delivery devices such as those described in U.S. Pat. Nos. 3,845,770; 3,916,899; 3,536,809; 3,598,123; and 4,008,719, the disclosures of which are incorporated herein by reference.

While the present invention has been described in terms of its specific embodiments, certain modifications and equivalents will be apparent to those skilled in the art and are included within the scope of the present invention. The examples are provided to illustrate particular aspects of the disclosure and do not limit the scope of the present invention as defined by the claims.

### EXAMPLES

### Example 1

### Preparation of 3-(4-pyridin-3-yl phenoxy) propan-1-amine

### Step 1: Preparation of pyridin-3-boronic Acid

3-bromo pyridine (5 g) was dissolved in about 20 mL dry tetrahydrofuran and cooled to −78° C. Tri-isopropyl borate (14.6 mL) was added to the solution followed by adding butyl lithium (15% in hexane, 21 mL). The reaction mixture was stirred at −78° C. for about 4 hours, pH was adjusted to about 7, it was extracted with ethyl acetate and concentrated to yield white solid.

Yield: 1.2 g

### Step 2: Preparation of 2-[3-(3-bromo-phenoxy)-propyl]-isoindole-1,3-dione

To 3-bromo phenol (0.03178 mol, 1 equiv.), about 30 mL of dry dimethylformamide was added and the solution was cooled to 0° C. Sodium hydride (0.0476 mol, 1.5 equiv.) was then added in portions. After 30 minutes, N-3-bromopropyl phthalimide (0.0381 mol, 1.2 equiv.) was added. The reaction mixture was stirred for about 3 hours and then quenched by pouring the reaction mixture into ice cold water. A precipitate thus formed was filtered and vacuum dried to yield the title compound.

Yield: 9 g

### Step 3: Preparation of 2-{3-[3-(pyridin-3-yl)-phenoxy]-propyl}-isoindole-1,3-dione

Pyridin-3-boronic acid (6.11 mmol, 1.1 equiv.), 2-[3-(3-bromo-phenoxy)-propyl]-isoindole-1,3-dione (5.5 mmol, 1 equiv.) and potassium carbonate (22.2 mmol, 4 equiv.) were added to a round bottom flask and degassed for about 1 hour. Dry dimethylformamide (15 mL) was added. The reaction mixture was then flushed with argon for about 15 minutes. Tetrakis(triphenylphosphine)palladium (0) (0.27 mol, 0.05 equiv.) was added to it. The reaction mixture was heated at 80° C. for about 12 hours, quenched with water and extracted with ethyl acetate. The organic layer was washed with water followed by brine and dried over anhydrous sodium sulfate.

Yield: 0.2 g

### Step 4: Preparation of 3-(4-pyridin-3-ylphenoxy)-propan-1-amine

2-{3-[3-(pyridin-3-yl)-phenoxy]-propyl}-isoindole-1,3-dione (0.2 g) was added to about 10 mL of ethanol followed by hydrazine monohydrate (0.2 mL). The reaction mixture was heated to and maintained at 60° C. for about 4 hours, cooled to room temperature and a solid thus obtained was filtered through a celite bed. The filtrate was concentrated to yield the crude product, which was purified using dichloromethane and methanol as eluent.

Yield: 0.13 g

The following compounds were prepared similarly:

### a)-3-[4-(3-Thienyl)phenoxy]propan-1-amine

The title compound was prepared by a procedure analogous to the one described step 3 by substituting pyridin-3-ylboronic acid with 3-thienylboronic acid

### b)-3-(Pyridin-3-yl-oxy)propan-1-amine

The title compound was prepared by a procedure analogous to the one described in step 2 by substituting 3-bromophenol with Pyridin-3-ol

### Example 2

### Preparation of 4-(3H-imidazo[4,5-b]pyridin-3-yl)butan-1-amine

4-(3H-imidazo[4,5-b]pyridin-3-yl)butan-1-amine was prepared by following the procedure disclosed in U.S. Pat. No. 5,635,485, which is incorporated herein in its entirety. In particular, 10.3 g of potassium carbonate were added to a solution of 5.95 g of 4-azabenzimidazole and 15.5 g of N-4-bromobutyl-phthalimide in 30 mL of dimethylformamide and the mixture was stirred for 20 hours at ambient temperature. The insoluble part was filtered off and rinsed with methylene chloride. The organic phase was washed with water, dried over magnesium sulfate and evaporated. The oily residue obtained was washed with petroleum ether followed by isopropyl ether to yield 16.3 g of a yellow solid, which was purified by column chromatography on silica, eluting with a methylene chloride:acetone mixture to yield 4.9 g of a first product having a melting point at 143° C.

A mixture of 32.86 g of the first product obtained above, 697 mL of ethanol and 20 mL of hydrazine was refluxed for 19 hours. The mixture was allowed to cool to ambient temperature, filtered, rinsed and evaporated to dryness. The residue was dissolved in methylene chloride, filtered, rinsed and evaporated to dryness to yield 18.87 g of the title compound.

### Example 3

### Preparation of 3-isoquinolin-5-ylpropanal

### Step I: Preparation of Dioxolane Derivative of Triphenylphosphorane

A solution of 2-bromomethyl-[1,3]dioxolane (60.0 mmol) and triphenylphosphine (60.0 mmol) in toluene (50 mL) was refluxed for 24 hours. A solid thus formed was filtered at room temperature, washed with diethyl ether and dried under reduced pressure to yield the title compound.

Yield: 3.5 g (54%)

M.P: 194-195° C.

### Step II: Preparation of 5-[2-(1,3-dioxolan-2-yl)vinyl]isoquinoline

A solution of the Wittig salt (1.97 mmol) obtained in Step I and isoquinoline-2-carboxyaldehyde (7.97 mmol) (commercially available, Biocom) in about 20 mL of tetrahydrofuran was cooled to −35° C., potassium-t-butoxide (9.9 mmol) was added in portions, and the mixture was stirred at −35° C. for about 1 hour. The mixture was then warmed to 10° C., stirred for about 3 hours, poured into water and extracted with dichloromethane. The organic layer was dried over anhydrous sodium sulfate, filtered and concentrated to yield the crude product, which upon purification over silica gel column using methanol-dichloromethane as eluent, yielded the title compound.

Yield: 1 g (55%).

### Step III: Preparation of 5-[2-(1,3-dioxolan-2-yl)ethyl]isoquinoline

5-[2-(1,3-dioxolan-2-yl)-vinyl] isoquinoline (100 mg) and Palladium/Carbon (50% wet, 50 mg) was added to a hydrogen bottle, flushed with argon and hydrogenated at 10 psi for about 12 hours. The mixture was filtered through a celite pad and the filtrate was evaporated to yield the title compound.

Yield: 70 mg (99%)

Step IV: Preparation of 3-isoquinolin-5-yl-propanal

A solution of 5-(2-[1,3-dioxolan-2-yl-ethyl] isoquinoline (0.9 g) and 2N Hydrochloric acid (15 mL) in acetone (15 mL) was stirred at 40° C. for 4 hours under nitrogen atmosphere. The solvent was evaporated off and the pH was adjusted to 9-10 using an aqueous potassium carbonate solution and extracted with ethyl acetate. Evaporation of ethyl acetate extract yielded the title compound.

Yield: 0.5 g (68%).

### Example 3a

### Preparation of 3-[4-(2-chloropyrimidin-5-yl)-1H-imidazol-1-yl]propanal

### Step 1: Preparation of 2-chloro-5-(1-trityl-H-imidazol-4-yl)pydimidine

A 3M solution of ethyl magnesium bromide in diethylether (12.66 mmol) was added to a solution of 4-iodo-1-trityl-1H-imidazole (10.55 mmol) in freshly distilled dry tetrahydrofuran (46.0 mL) in a three necked round bottom flask at room temperature. The reaction mixture was stirred for about 90 minutes and then a 1M solution of zinc chloride (12.66 mmol) was added at room temperature. The solution was stirred for about 90 minutes and then degassed for about 20.0 minutes. Palladium triphenylphosphine (0.61 g, 0.527 mmol) and 5-bromo-2-chloro pyrimidine (11.6 mmol) were added and the reaction mixture was stirred for about 12-14 hrs at about 70° C. The reaction mixture was cooled, diluted with dichloromethane (100.0 mL), washed with aqueous solution of ethylenediaminetetraacetic acid (pH 9), brine, dried over anhydrous sodium sulfate, filtered and concentrated to yield a solid, which was purified by column using ethyl acetate-hexane as eluent to yield the title compound.

Yield: 3.0 g (67%).

Step 2: Preparation of 2-chloro-5-(1H-imidazol-4-yl)pyrimidine

Concentrated hydrochloric acid (2.0 mL) was added to a solution of 2-chloro-5-(1-trityl-1H-imidazol-4-yl)-pyrimidine (6.2 g) in ethanol (62 mL) and the solution was heated to and maintained at about 45° C. for about 3 hours. The reaction mixture was cooled to about 30° C. and ethanol was evaporated under reduced pressure. The resulting residue was basified using aqueous saturated sodium bicarbonate solution (pH 7.5) and then extracted with ethyl acetate (150.0 mL) twice. All organic layers were then combined and washed with water followed by brine, dried over anhydrous sodium sulphate and concentrated. The resulting crude product was purified by column chromatography to yield the title compound.

Yield: 2.7 g.

### Step 3: Preparation of 3-[4-(2-chloropyrimidin-5-yl)-1H-imidazol-1-yl]proanal

Acrolein (4 equiv. 0.74 mL, 11.11 mmol) was added to a mixture of 2-Chloro-5-(1H-imidazol-4-yl)-pyrimidine (1 eq., 500 mg, 2.78 mmol) and ethanol (1 eq., 0.16 mL, 2.73 mmol) in tetrahydrofuran (10 mL). The reaction mixture was stirred at about 80° C. for about 7 hours and then quenched by adding a few drops of water. The solvent was evaporated and the solid thus obtained was adsorbed over silica gel (100-200 mesh) and purified by column chromatography using dichloromethane and methanol as eluent to yield the title compound.

Yield: 500 mg.

### Example 4

### Preparation of 3-Pyrrolo[2,3-b]pyridine-1-yl-propionaldehyde

1H-Pyrrolo[2,3-b]pyridine (8.403 mmol) (commercially available, Lancaster) was dissolved in tetrahydrofuran (30 mL) and ethanol (8.403 mmol) and acrolein (33.612 mmol) were added. The reaction mixture was stirred at 60° C. for about 6 hours. Volatiles were removed and the residue thus obtained was purified over silica gel column using methanol and dichloromethane as eluent to yield the title compound.

Yield: 31%

The following compounds were prepared similarly:

### a)-3-(4-phenyl-imidazol-1-yl)-propionaldehyde

The title compound was prepared by a procedure analogous to the one described above by substituting 1H-Pyrrolo[2,3-b]pyridine with 4-phenyl-1H-imidazole (commercially available, Lancaster)

### b)-3-(1-H-Benzimidazol-1-yl)propanal

The title compound was prepared by a procedure analogous to the one described above by substituting 1H-Pyrrolo[2,3-b]pyridine with 1-H-Benzimidazol (commercially available, Loba Chemie)

### c)-3-(4-Pyridin-3-yl-1H-imidazol-1-yl)propanal

The title compound was prepared by a procedure analogous to the one described above by substituting 1H-Pyrrolo[2,3-b]pyridine with 4-Pyridin-3-yl-1H-imidazole (commercially available, Argus Chemicals)

### d)-3-[4-(3-thienyl)-1H-imidazol-1-yl]propanal

The title compound was prepared by a procedure analogous to the one described above by substituting 1H-Pyrrolo[2,3-b]pyridine with 3-Thienyl-1H-imidazole

### Example 5

### Preparation of 4-(thienyl-3-yl)imidazole

Step 1: A solution of 1-trityl-4-bromo-imidazole (10.28 mmol), thiophene-3-boronic acid (12.33 mmol) and potassium carbonate (41.12 mmol) in dimethylformamide (50 mL) was degassed for 15 minutes with stirring at room temperature followed by adding tetrakis(triphenylphospine)palladium (1.028 mmol). The reaction mixture was stirred at 90° C. for about 20 hours, cooled, poured into water and extracted with ethyl acetate. The solvent was evaporated to yield a crude product, which was purified by column using ethylacetate:hexane (˜15%) as eluent.

Yield: 2.45 g

Step 2: The product of the coupling reaction (0.00625 mol) obtained from step 1 above was added to ethanol, then hydrochloric acid (1N, 1 mL) was added and the resulting solution was heated to and maintained at 50° C. for about 2 hours. The reaction mixture was then cooled to room temperature, the solvent was evaporated and pH was adjusted to 8 using sodium bicarbonate solution. The product was extracted with ethyl acetate and evaporation of ethyl acetate yielded the crude product, which was purified by column chromatography using dichloromethane:methanol as the eluent to yield the title compound.

Yield: 0.81 g

### Example 6

### Preparation of Compound of Formula III

To an aqueous solution of hydrochloric acid (1N, 250 mL) was added clarithromycin (33.4 mmol) portionwise at ambient temperature. The reaction mixture was neutralized with solid sodium bicarbonate and the aqueous layer was extracted with ethyl acetate. The organic layer was washed with water followed by brine, and dried over anhydrous sodium sulphate. The solvent was removed under reduced pressure to afford crude product, which was recrystallized by using ethyl acetate and hexane to yield the title compound.

### Example 7

### Preparation of Compound of Formula IV

To a solution of compound of Formula III (1 equiv.) in dry dichloromethane was added benzoic anhydride (2.5 equiv.) followed by triethylamine (6 equiv.). The mixture was stirred at an ambient temperature for about 30 hours and then quenched by adding sodium bicarbonate solution. The aqueous layer was extracted with dichloromethane, washed successively with water, brine, and dried over anhydrous sodium sulfate. The solvent was removed under reduced pressure to yield a crude product, which was recrystallized by using ethyl acetate and hexane mixture to form the title compound.

### Example 8

### Preparation of Compound of Formula V

To a solution of compound of Formula IV (1 equiv.) in dichloromethane at about 0° C. was added triphosgene (1.5 equiv.) with stirring. Pyridine (15 equiv.) was then slowly added to the mixture and an exothermic reaction was observed. After complete addition, the reaction mixture was stirred for about 3-4 hours at 0° C. The reaction mixture was then quenched by dropwise addition of ice-cold water, diluted with dichloromethane and washed with water followed by brine, dried over anhydrous sodium sulphate and concentrated under reduced pressure to yield the title compound.

### Example 9

### Preparation of Compound of Formula VI

To a solution of compound of Formula V (1 equiv.) in dimethylformamide was added tetramethyl guanidine (2.2 equiv.) and the mixture was heated to and maintained at about 80-90° C. for about 8 hours. The reaction mixture was cooled to ambient temperature, water was added and the mixture was extracted with ethyl acetate, washed with water followed by brine, dried over anhydrous sodium sulfate and concentrated under reduced pressure to yield the title compound.

### Example 10

### Preparation of Compound of Formula VII

To a solution of compound of Formula VI (1 equiv.) in dichloromethane was added Dess-Martin Periodinane (2.5 equiv.) and the reaction mixture was stirred at 30° C. for about 1 hour. The reaction mixture was quenched by adding saturated aqueous potassium carbonate solution followed by saturated sodium thiosulphate solution and the resulting mixture was stirred. The reaction mixture was then separated and extracted with dichloromethane, the dichloromethane layer was washed with water followed by brine, dried over anhydrous sodium sulphate and concentrated under reduced pressure to yield the title compound.

### Example 11

### Preparation of Compound of Formula VIII

To a solution of compound of Formula VII (1 equiv.) in dry acetonitrile:dichloromethane (2:1) was added N-iodosuccinimide (2 equiv.). The reaction mixture was allowed to attain ambient temperature and then it was stirred for about 3-4 hours. Sodium bisulphite solution was then added and stirred followed by adding sodium carbonate solution with stirring. Dichloromethane was evaporated under reduced pressure and the residue thus obtained was extracted with ethyl acetate, washed with water then brine, dried over anhydrous sodium sulfate and then the solvent was removed under reduced pressure to yield the title compound.

### Example 12

### Preparation of Compound of Formula IX

To a solution of compound of Formula VIII (1 equiv.) in acetonitrile was added solid sodium hydrogen carbonate (5 equiv.) and R³X (6 equiv.) under argon at ambient temperature and the mixture was stirred for about 18-20 hours. The reaction mixture was quenched by adding water. The reaction mixture was diluted with ethyl acetate and washed with water followed by brine, dried over anhydrous sodium sulfate and concentrated under reduced pressure to yield a crude product. The crude product was purified by silica gel column chromatography (thoroughly neutralized with triethylamine) using 10-15% acetone in hexane to yield the title compound.

### Example 13

### Preparation of Compound of Formula X

Method A: To a solution of compound of Formula IX (1 equiv.) in dimethylformamide was added sodium hydride (1.5 equiv.) at 0° C. The mixture was stirred for about 15 minutes and then N-fluorobenzene sulfonimide (1.2 equiv.) was added. The reaction mixture was stirred at 0° C. for about 3 hours, quenched by adding water and extracted with ethyl acetate. The organic layer was washed with water followed by brine, dried over anhydrous sodium sulfate and concentrated under reduced pressure.
Method B: To a solution of compound of Formula IX (1 equiv.) in tetrahydrofuran was added potassium-t-butoxide at about −15° C. and the mixture stirred for about 20 minutes. N-fluorobenzene sulfonimide (1.2 equiv.) in tetrahydrofuran was then added and the reaction mixture was stirred at −15° C. for about 2 hours, quenched by adding water and extracted with ethyl acetate. The organic layer was washed with water followed by brine, dried over anhydrous sodium sulphate and concentrated under reduced pressure.

### Example 14

### Preparation of Compound of Formula XI

A solution of compound of Formula X (1 equiv.) in dimethylformamide:tetrahydrofuran (3:2) was cooled to 0° C., N,N′-carbonyldiimidazole (3 equiv.) and sodium hydride (3 equiv.) were added and the mixture was stirred for about 30 minutes. The reaction mixture was quenched by adding water and then extracted with ethyl acetate. The ethyl acetate layer was washed with water followed by brine, dried over anhydrous sodium sulphate and concentrated under reduced pressure to yield the title compound.

### Example 15

### Preparation of Compound of Formula XII

A compound of Formula XI (1 equiv.) and compound of Formula R-W-NH₂ (3 equiv.) were added to 10% water in acetonitrile and the mixture was heated to and maintained at 65-70° C. for about 14 hours. The reaction mixture was cooled to ambient temperature and acetonitrile-water was removed under reduced pressure. The resulting residue was purified by silica gel column chromatography (thoroughly neutralized with triethylamine) using 25-30% acetone in hexane to yield the title compound.

### Example 16

### Preparation of Compound of Formula XIII

A compound of Formula XII was added to methanol and refluxed for about 12 hours. The reaction mixture was cooled to ambient temperature and methanol was evaporated under reduced pressure. The resulting solid mass was purified over silica gel column using 2-6% methanol in dichloromethane to form the title compound.

The following compounds were prepared following the above general procedure:
Compound No. 2: 2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(3-pyridin-3-yl-phenoxy)-propyl)-imino)]erythromycin A; Mass: m/z 856.40 [M+1],
Compound No. 3: 2-α-Fluoro-5-O-(3′-N-desmethyl-3′-N-allyl)-11,12-dideoxy-3-O-decladinosyl-6-O-methyl-3-oxo-12,11-[oxycarbonyl-((3-(3-pyridin-3-yl-phenoxy)-propyl)-imino)]erythromycin A; Mass: m/z 868.40 [M+1],
Compound No. 4: 2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(pyridin-3-yloxy)-propyl)-imino)]erythromycin A; Mass: m/z 780.47 [M+1],
Compound No. 5: 2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(3-thiophen-3-yl-phenoxy)-propyl)-imino)]erythromycin A; Mass: m/z 861.82 [M+1].

### Example 17

### Preparation of Compound of Formula XIIIA

To a solution of compound of Formula XII (1 equiv.) in dry acetonitrile:dichloromethane (2:1) was added N-iodosuccinimide (2 equiv.) at 0° C., the reaction mixture was allowed to attain ambient temperature and the mixture was stirred for about 3-4 hours. A sodium bisulphite solution was added and stirred followed by adding a sodium carbonate solution with stirring. Dichloromethane was evaporated under reduced pressure and the residue thus obtained was extracted with ethyl acetate, washed with water followed by brine, dried over anhydrous sodium sulfate and the solvent was removed under reduced pressure to yield the title compound.

### Example 18

### Preparation of Compound of Formula XIVA

A compound of Formula XIIIA was added to methanol and refluxed for about 12 hours. The reaction mixture was cooled to ambient temperature and methanol was evaporated under reduced pressure. The resulting solid mass was purified over a silica gel column using 2-6% methanol in dichloromethane to yield the title compound.

The following compound was prepared following the above general procedure
Compound No. 1: 2-α-Fluoro-5-O-(3′-N-didesmethyl-3′-N-ethyl)-11,12-dideoxy-3-O-decladinosyl-6-O-methyl-3-oxo-12,11-[oxycarbonyl-((4-imidazol[4,5-b]pyridin-3-yl)-butyl)-imino]erythromycin A; Mass: m/z 804.30 [M+1].

### Example 19

### Preparation of Compound of Formula XIV

Step 1: To the compound of Formula XI (15 mmol) in dimethylformamide (10 mL) was added hydrazine hydrate (6.0 mmol) and the mixture stirred for about 1.5 hours at room temperature. The reaction mixture was quenched by pouring the mixture into water and then extracted with ethyl acetate. The organic layer was concentrated, washed with brine to yield carbazate epimers.
Step 2: A solution of epimers obtained in Step 1 above (1.308 mmol) in tetrahydrofuran (10 mL) was cooled to about 0° C., potassium-t-butoxide (2.354 mmol) was added and the mixture stirred for about 2 hours. The reaction mixture was quenched by pouring the mixture into water and then extracted with ethyl acetate. Ethyl acetate was then evaporated to yield the crude product of the desired isomer of Formula XIV. This crude product was purified by column chromatography of silica gel using hexane:acetone and 2% triethylamine as the eluting solvent to yield the title compound.

### Example 20

### Preparation of Compound of Formula XV

A compound of Formula XIV was stirred in methanol (20 mL methanol per 1 g of compound) at 65-70° C. for about 24-48 hours. The solvent was evaporated and a solid thus obtained was purified by column chromatography using 100-200 mesh silica gel and dichloromethane:methanol as the eluting solvent to yield the title compound.

### Example 21

### Preparation of Compound of Formula XVI

A compound of Formula XV, R-W-CHO (5 equiv.) and acetic acid (5 equiv.) were added to methanol (20 mL methanol per 1 g of compound) and stirred at room temperature for about 2 hours. Acetic acid (5 equiv.) and sodium cyanoborohydride (5 equiv.) were then added and the mixture was stirred for 16-24 hours. The solvent was evaporated and the residue thus obtained was purified by column chromatography using silica gel and hexane:acetone+2% triethylamine as the eluting solvent to yield the title compound.

The following compounds were prepared following the above general procedure
Compound No. 6: 2-α-Fluoro-5-O-(3′-N-desmethyl-3′-N-ethyl)-11,12-dideoxy-3-O-decladinosyl-6-O-methyl-3-oxo-12,11-[oxycarbonyl-hydrazo]erythromycin A; Mass: m/z 660.46 [M+1],
Compound No. 7: 2-α-Fluoro-5-O-(3′-N-desmethyl-3′-N-allyl)-11,12-dideoxy-3-O-decladinosyl-6-O-methyl-3-oxo-12,11-[oxycarbonyl-hydrazo]erythromycin A; Mass: m/z 672.49 [M+1],
Compound No. 8: 2-α-Fluoro-5-O-(3′-N-desmethyl-3′-N-allyl)-11,12-dideoxy-3-O-decladinosyl-6-O-methyl-3-oxo-12,11-[oxycarbonyl-((3-pyrrolo[2,3-b]pyridin-1-yl)-propyl)-hydrazo)]erythromycin A; Mass: m/z 830.38 [M+1],
Compound No. 9: 2-α-Fluoro-5-O-(3′-N-desmethyl-3′-N-allyl)-11,12-dideoxy-3-O-decladinosyl-6-Q-methyl-3-oxo-12,11-[oxycarbonyl-((3-phenylpropyl)-hydrazo)]erythromycin A; Mass: m/z 790.45 [M+1],
Compound No. 10: 2-α-Fluoro-5-O-(3′-N-desmethyl-3′-N-allyl)-11,12-dideoxy-3-O-decladinosyl-6-O-methyl-3-oxo-12,11-[oxycarbonyl-((3-isoquinolin-5-yl-propyl-hydrazo)]erythromycin A; Mass: m/z 841.36 [M+1],
Compound No. 11: 2-α-Fluoro-5-O-(3′-N-desmethyl-3′-N-ethyl)-11,12-dideoxy-3-O-decladinosyl-6-O-methyl-3-oxo-12,11-[oxycarbonyl-((3-phenylpropyl)-hydrazo)]erythromycin A; Mass: m/z 778.76 [M+1],
Compound No. 12: 2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-phenyl-imidazol-1-yl)-propyl)-hydrazo)]erythromycin A; Mass: m/z 856.64 [M+1],
Compound No. 13: 2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-phenyl-imidazol-1-yl)-propyl)-hydrazo)]erythromycin A; Mass: m/z 844.52 [M+1],
Compound No. 14: 2-α-Fluoro-5-O-(3′-N-desmethyl-3′-N-ethyl)-11,12-dideoxy-3-O-decladinosyl-6-O-methyl-3-oxo-12,11-[oxycarbonyl-((3-benzoimidazol-1-yl)-propyl)-hydrazo)]erythromycin A; Mass: m/z 818.48 [M+1],
Compound No. 15: 2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-pyridyl-3-yl-imidazol-1-yl)-propyl)-hydrazo)]erythromycin A; Mass: m/z 845.45 [M+1],
Compound No. 16: 2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-pyridyl-3-yl-imidazol-1-yl)-propyl)-hydrazo)]erythromycin A; Mass: m/z 857.45 [M+1],
Compound No. 17: 2-α-Fluoro-5-O-(3′-N-desmethyl-3′-N-allyl)-11,12-dideoxy-3-O-decladinosyl-6-O-methyl-3-oxo-12,11-[oxycarbonyl-((3-benzoimidazol-1-yl)-propyl)-hydrazo)]erythromycin A; Mass: m/z 830.41 [M+1],
Compound No. 18: 2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-thiophen-3-yl-imidazol 1-yl)propyl)-hydrazo)]erythromycin A; Mass: m/z 862.51 [M+1],
Compound No. 22: 2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-pyridyl-3-yl-imidazol-1-yl)-butyl)-hydrazo)]erythromycin A; Mass: m/z 871.34 [M+1],
Compound No. 23: 2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-phenyl-imidazol-1-yl)-butyl)-hydrazo)]erythromycin A; Mass: m/z 870.44 [M+1],
Compound No. 24: 2-α-Fluoro-5-O-(3′-N-desmethyl-3′-N-ethyl)-11,12-dideoxy-3-O-decladinosyl-6-O-methyl-3-oxo-12,11-[oxycarbonyl-((3-phenyl-allyl)-hydrazo)]erythromycin A; Mass: m/z 776.73 [M+1],
Compound No. 25: 2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-(2-chloro-pyrimidin-5-yl)-imidazol-1-yl)-propyl)-hydrazo)]erythromycin A; Mass: m/z 880.9 [M+1].

### Example 22

### Preparation of Compound of Formula XVII

A compound of Formula XV (0.742 mmol) and hydrochloride salt of H₂NNOR¹¹ (14.84 mmol) in ethanol (6 mL) was stirred at reflux temperatures for about 40 hours. The solvent was evaporated, dichloromethane was added and the pH was adjusted to about 9.0 by adding 1 N sodium hydroxide. The aqueous layer was extracted with dichloromethane and the combined organic layers were washed with brine, dried and concentrated to yield the title compound.

Yield: 0.430 g

### Example 23

### Preparation of Compound of Formula XVIII

A compound of Formula XVII (0.624 mmol) and R-W-CHO (1.248 mmol) were added to methanol (10 mL), glacial acetic acid (53.62 mmol) was then added to the mixture at room temperature and the mixture was stirred for about one hour. Acetic acid (53.62 mmol) followed by sodium cyanoborohydride (3.12 mmol) were then added and the reaction mixture was stirred for about 12-18 hours. The solvent was removed under reduced pressure and the residue thus obtained was extracted with ethyl acetate and purified by column chromatography using silica gel and acetone:hexane:2% triethylamine as eluent to yield the title compound.

Yield: 0.120 g

The following compounds were prepared following the above general procedure
Compound No. 19: 2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-phenyl-imidazol-1-yl)-propyl)-hydrazo)]erythromycin A-9-(O-methyl)oxime; Mass: m/z 885.59 [M+1],
Compound No. 20: 2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-pyridyl-3-yl-imidazol 1-yl)-propyl)-hydrazo)]erythromycin A-9-(O-methyl)oxime; Mass: m/z 874.41 [M+1],
Compound No. 21: 2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-thiophen-3-yl-imidazol 1-yl)propyl)-hydrazo)]erythromycin A-9-(O-methyl)oxime; Mass: m/z 891.55 [M+1].

### Example 24

### Microbiological Activity

Compounds disclosed herein displayed antibacterial activity in vitro especially against strains, which are resistant to macrolides either due to efflux (mef strains) or ribosomal modification (erm) strains. These compounds are useful in the treatment of community-acquired pneumonia, upper and lower respiratory tract infections, skin and soft tissue infections, hospital-acquired lung infections, bone and joint infections, and other bacterial infections, for example, mastitis, catheter infection, foreign body, prosthesis infections or peptic ulcer disease.

Minimum inhibitory concentration (MIC) has been an indicator of in vitro antibacterial activity widely used in the art.

### Procedure

### Medium

a) Cation adjusted Mueller Hinton Agar (MHA-Difco)

### b) Trypticase Soya Agar (TSA)

### Inoculum Preparation

The cultures were streaked on TSA for aerobic cultures and MHA with 5% sheep blood for fastidious cultures. Aerobic cultures were incubated at 37° C. for about 18-24 hours. Fastidious cultures were incubated CO₂ incubation (5% CO₂) at 37° C. for about 18-24 hours. Three to four well isolated colonies were taken and saline suspensions were prepared in sterile densimat tubes. The turbidity of the culture was adjusted to 0.5-0.7 McFarland standard (1.5×10 CFU/mL (Colony Forming Unit)/mL). The cultures were diluted 10 fold in saline to form inoculum size of approximately 1-2×10⁷ organisms/mL.

### Preparation of Drug Concentration

1 mg/mL concentration of stock solution of drugs was prepared in dimethyl sulphoxide/distilled water/solvent given in National Committee for Clinical Laboratory Standards (NCCLS) manual. Serial two-fold dilutions of the compounds and standard drugs were prepared as per NCCLS manual.

Stock solution can be changed according to the need of the experiment.

### Preparation of Agar Plates

2 mL of respective drug concentration was added to 18 mL of Molten Mueller Hinton agar to achieve the required range, for example 0.015 μg/mL-16 μg/mL. For fastidious culture added 1 mL of sheep blood in Molten Mueller Hinton agar.

For control MHA and MHA with 5% sheep blood plates without antibiotic for each set were prepared. One MHA and MHA with 5% sheep blood plates without antibiotic for determining quality check for media was prepared.

### Preparation of Teflon Template

1 μg of each culture on each plate was replicated with the help of replicator (Denley's multipoint replicator). The spots were allowed to dry and the plates were incubated for about 18-24 hours at 37° C. Fastidious cultures were incubated at 37° C. in CO₂ incubator. The results were noted with the control plates.

### Endpoint Definition

The concentration of drug at which there was complete disappearance of growth spot or formation of less than 10 colonies per spot was considered as Minimum Inhibitory Concentration (MIC).

The MICs of quality control (QC) strains were plotted on the QC chart for agar dilution method. If the MICs were within the range, the results interpreted by comparing MICs of standards against all organisms with those of test compounds.

### Precautions & Quality Control Measures

### Quality Control Strains

Staphylococcus aureus ATCC 29213
Enterococcus faecalis ATCC 29212
Eschericia coli ATCC 25922
Pseudomonas aeruginosa ATCC 27853
All 60 cultures were visually checked for purity.

Media Control: Performed NCCLS disc diffusion assay using 10 μg discs of Gentamicin (Difco) against Ps. aeruginosa ATCC 27853. A zone diameter of 16-21 mm should be considered for optimum cation (Magnesium and Calcium) content of the media. Plotted the diameter in the media QC chart.

Results: The compounds disclosed herein were found to be active against staphylococci, enterococci, Moraxella catarrhalis, Streptococcus pnemoniae, Streptococcus pyogenes, Haemophilus influenzae strains. MIC of the described compounds were:
a) from about 0.004 to about 16 μg/mL, from about 0.0125 to about 8 μg/mL, and even from about 0.015 to about 4 μg/mL for Steptococcus pnemoniae;
b) from about 0.004 to about 16 μg/mL, from about 0.015 to about 8 μg/mL, even from about 0.03 to about 4 μg/mL for Streptococcus pyogenes;
c) from about 0.06 to about 8 μg/mL, from about 0.06 to about 4 μg/mL, even from about 0.125 to about 2 μg/mL for Haemophilus influenzae;
d) from about 0.03 to about 4 μg/mL, from about 0.03 to about 1 μg/mL, even from about 0.125 to about 0.5 μg/mL for Moraxella catarrhalis;
e) from about 0.125 to about 16 μg/mL, from about 0.125 to about 0.5 μg/mL, and even from about 0.125 to about 0.25 μg/mL for staphylococci; and
f) from about 0.06 to about 16 μg/mL, 0.06 to about 8 μg/mL, even from about 0.125 to about 0.5 μg/mL for enterococci.

Some compounds described herein exhibited good activity against erythromycin resistant and telithromycin resistant Streptococcus pyogenes 1721 erm B

### REFERENCES

National Committee for Clinical Laboratory Standards (NCCLS), Methods for Dilution Antimicrobial Susceptibility Tests for Bacteria That Grow Aerobically—Fifth Edition; Approved Standard. M7-A5, Vol. 20. No. 2 (January 2000).
National Committee for Clinical Laboratory Standards, Performance Standards for Antimicrobial Susceptibility Testing—Twelfth informational supplement, M 100-S12, Vol. 22 No. 1 (January 2002).

We claim:

1. A compound having the structure of Formula I, 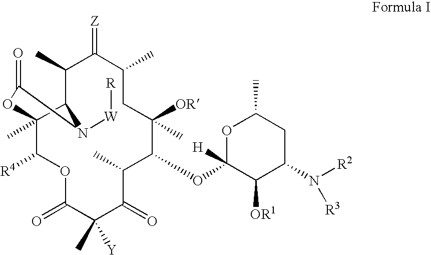
pharmaceutically acceptable salts, pharmaceutically acceptable solvates, stereoisomers, prodrugs, metabolites and polymorphs thereof, wherein:
R¹ is hydrogen, hydroxyl protecting group;
R² and R³ are independently hydrogen, alkyl, alkenyl, alkynyl, cycloalkyl, aryl, (heterocyclyl)alkyl or —COR¹¹, wherein
R¹¹ is hydrogen, alkyl, aryl, NR⁹R¹⁰ or alkoxy;
R⁹ and R¹⁰ are independently hydrogen, alkyl, alkenyl or alkynyl; and
with the proviso that R² and R³ are not simultaneously methyl;
W is —NH or —(CH₂)ₘ—, wherein
m is an integer of from 2 to 6;
—(CH₂)ₘ— group is optionally interrupted by one or more of unsaturated bond, oxygen, sulfur, —NRₐ— or combination thereof, wherein
Rₐ is hydrogen, alkyl, cycloalkyl, alkenyl, heterocyclyl, (heterocyclyl)alkyl, alkynyl or aryl;
one of the hydrogen atoms of —(CH₂)ₘ— group is optionally replaced by halogen, alkyl, hydroxyl or alkoxy; and
with the proviso that R² is hydrogen when W is —(CH₂)ₘ—, wherein m is an integer of from 2 to 6;
R is hydrogen, hydroxy, alkyl, aryl, heterocyclyl, cycloalkyl or cycloalkenyl;
R⁴ is hydrogen, alkyl, alkenyl, alkynyl, aryl, cycloalkyl, heterocyclyl, amidoarylalkyl or amidoarylalkynyl;
R′ is hydrogen, aryl, alkyl or —(CH₂)ᵣ-U; wherein
r is an integer of from 1 to 4; and
U is alkenyl, alkynyl, alkylalkenyl, alkylalkynyl, arylalkenyl, arylalkynyl, heterocyclylalkenyl or heterocyclylalkynyl; or
OR′ is replaced by hydrogen;
Y is hydrogen, halogen, cyano, alkyl, aryl, heterocyclyl, hydroxy, amino, PhSe, alkenyl, alkynyl or —NR⁹R¹⁰, wherein
R⁹ and R¹⁰ are the same as defined earlier; and
Z is oxygen, sulphur or NOR¹¹, wherein
R¹¹ is the same as defined earlier.

2. The compound of claim 1, wherein R¹ is hydrogen; R² is hydrogen or alkyl; R⁴ is alkyl; Y is halogen; R³ is alkyl or alkenyl; R′ is alkyl; Z is oxygen or NOR¹¹; W is —NH or —(CH₂)₄—, wherein the —(CH₂)₄— group is interrupted by oxygen, nitrogen or unsaturated bond or one of the hydrogen atoms of —(CH₂)₄— group is replaced by alkyl; and R is hydrogen, aryl, substituted aryl or heterocyclyl.

3. The compound of claim 1, wherein R¹ is hydrogen; R² is hydrogen or methyl; R⁴ is ethyl; Y is fluorine; R³ is ethyl or allyl; R′ is methyl; Z is oxygen or —NOCH₃; W is —NH—, —(CH₂)₃O—, —NH—(CH₂)₃—, —NHCH₂CH═CH— or —NH(CH₂)₂—CH(CH₃)— and R is phenyl, 3-(pyridine-3-yl)-phenyl, 3-(thienyl-3-yl)-phenyl, pyridin-3-yl, imidazo[4,5b]pyridin-3-yl, pyrrolo[2,3b]pyridin-1-yl, isoquinoline-5-yl and benzimidazol-1-yl, 3-(1H-imidazol-4-yl)-pyridine, 4-phenyl-1H-imidazole or 4-thiophen-3-yl-1H-imidazole.

4. A compound selected from:
2-α-Fluoro-5-O-(3′-N-didesmethyl-3′-N-ethyl)-11,12-dideoxy-3-O-decladinosyl-6-O-methyl-3-oxo-12,11-[oxycarbonyl-((4-imidazol[4,5-b]pyridin-3-yl)-butyl)-imino]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(3-pyridin-3-yl-phenoxy)-propyl)-imino)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(3-pyridin-3-yl-phenoxy)-propyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(pyridin-3-yloxy)-propyl)-imino)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(3-thiophen-3-yl-phenoxy)-propyl)-imino)]erythromycin A,
2-α-Fluoro-5-O-(3-N-desmethyl-3-N-ethyl)-11,12-dideoxy-3-O-decladinosyl-6-O-methyl-3-oxo-12,11-[oxycarbonyl-hydrazo]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-hydrazo]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-pyrrolo[2,3-b]pyridin-1-yl)-propyl)-hydrazo)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-phenylpropyl)-hydrazo)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-isoquinolin-5-yl-propyl-hydrazo)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-phenylpropyl)-hydrazo)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-phenyl-imidazol-1-yl)-propyl)-hydrazo)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-phenyl-imidazol-1-yl)-propyl)-hydrazo)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-benzoimidazol-1-yl)-propyl)-hydrazo)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-pyridyl-3-yl-imidazol-1-yl)-propyl)-hydrazo)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-pyridyl-3-yl-imidazol-1-yl)-propyl)-hydrazo)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-benzoimidazol-1-yl)-propyl)-hydrazo)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-thiophen-3-yl-imidazol-1-yl)propyl)-hydrazo)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-phenyl-imidazol-1-yl)-propyl)-hydrazo)]erythromycin A-9-(O-methyl)oxime,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-pyridyl-3-yl-imidazol-1-yl)-propyl)-hydrazo)]erythromycin A-9-(O-methyl)oxime,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-thiophen-3-yl-imidazol-1-yl)propyl)-hydrazo)]erythromycin A-9-(O-methyl)oxime,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-pyridyl-3-yl-imidazol-1-yl)-butyl)-hydrazo)]erythromycin A,
2-α-Fluoro-5-O-(3′-N-desmethyl-3′-N-allyl)-11,12-dideoxy-3-O-decladinosyl-6-O-methyl-3-oxo-12,11-[oxycarbonyl-((3-(4-phenyl-imidazol-1-yl)-butyl)-hydrazo)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-phenyl-allyl)-hydrazo)]erythromycin A,
2-α-Fluoro-5-O-(3′-N-desmethyl-3′-N-ethyl)-11,12-dideoxy-3-O-decladinosyl-6-O-methyl-3-oxo-12,11-[oxycarbonyl-((3-(4-(2-chloro-pyrimidin-5-yl)-imidazol-1-yl)-propyl)-hydrazo)]erythromycin A,
pharmaceutically acceptable salts, pharmaceutically acceptable solvates, stereoisomers, prodrugs, metabolites or polymorphs thereof.

5. A pharmaceutical composition comprising a therapeutically effective amount of one or more compounds having the structure of Formula I, 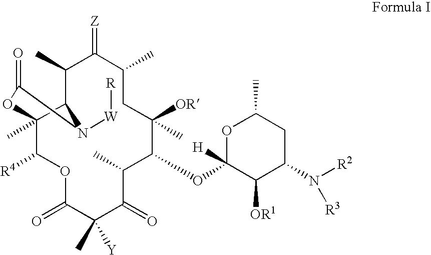
pharmaceutically acceptable salts, pharmaceutically acceptable solvates, stereoisomers, prodrugs, metabolites or polymorphs thereof, and optionally together with one or more pharmaceutically acceptable carriers, excipients or diluents,
wherein:
R¹ is hydrogen, hydroxyl protecting group;
R² and R³ are independently hydrogen, alkyl, alkenyl, alkynyl, cycloalkyl, aryl, (heterocyclyl)alkyl or —COR¹¹, wherein
R¹¹ is hydrogen, alkyl, aryl, NR⁹R¹⁰ or alkoxy;
R⁹ and R¹⁰ are independently hydrogen, alkyl, alkenyl or alkynyl; and
with the proviso that R² and R³ are not simultaneously methyl;
W is —NH or —(CH₂)ₘ—, wherein
m is an integer of from 2 to 6;
—(CH₂)ₘ— group is optionally interrupted by one or more of unsaturated bond, oxygen, sulfur, —NRₐ— or combination thereof, wherein
Rₐ is hydrogen, alkyl, cycloalkyl, alkenyl, heterocyclyl, (heterocyclyl)alkyl, alkynyl or aryl;
one of the hydrogen atoms of —(CH₂)ₘ— group is optionally replaced by halogen, alkyl, hydroxyl or alkoxy; and
with the proviso that R² is hydrogen when W is —(CH₂)ₘ—, wherein m is an integer of from 2 to 6;
R is hydrogen, hydroxy, alkyl, aryl, heterocyclyl, cycloalkyl or cycloalkenyl;
R⁴ is hydrogen, alkyl, alkenyl, alkynyl, aryl, cycloalkyl, heterocyclyl, amidoarylalkyl or amidoarylalkynyl;
R′ is hydrogen, aryl, alkyl or —(CH₂)ᵣ-U; wherein
r is an integer of from 1 to 4; and
U is alkenyl, alkynyl, alkylalkenyl, alkylalkynyl, arylalkenyl, arylalkynyl, heterocyclylalkenyl or heterocyclylalkynyl; or
OR′ is replaced by hydrogen;
Y is hydrogen, halogen, cyano, alkyl, aryl, heterocyclyl, hydroxy, amino, PhSe, alkenyl, alkynyl or —NR⁹R¹⁰, wherein
R⁹ and R¹⁰ are the same as defined earlier; and
Z is oxygen, sulphur or NOR¹¹, wherein
R¹¹ is the same as defined earlier.

6. A method for treating or preventing a condition caused by or contributed to by bacterial infection in a mammal comprising administering to the mammal in need thereof a pharmaceutical composition of claim 5.

7. A method for treating or preventing a condition caused by or contributed to by bacterial infection in a mammal comprising administering to the mammal in need thereof a therapeutically effective amount of one or more compounds having the structure of Formula I, 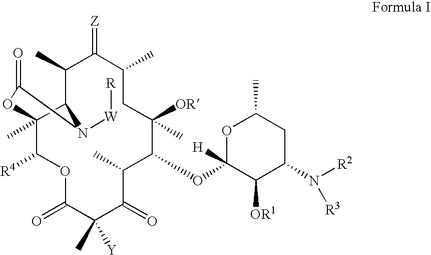
pharmaceutically acceptable salts, pharmaceutically acceptable solvates, stereoisomers, prodrugs, metabolites or polymorphs thereof, and optionally together with one or more pharmaceutically acceptable carriers, excipients or diluents,
wherein:
R¹ is hydrogen, hydroxyl protecting group;
R² and R³ are independently hydrogen, alkyl, alkenyl, alkynyl, cycloalkyl, aryl, (heterocyclyl)alkyl or —COR¹¹, wherein
R¹¹ is hydrogen, alkyl, aryl, NR⁹R¹⁰ or alkoxy;
R⁹ and R¹⁰ are independently hydrogen, alkyl, alkenyl or alkynyl; and
with the proviso that R² and R³ are not simultaneously methyl;
W is —NH or —(CH₂)ₘ—, wherein
m is an integer of from 2 to 6;
—(CH₂)ₘ— group is optionally interrupted by one or more of unsaturated bond, oxygen, sulfur, —NRₐ— or combination thereof, wherein
Rₐ is hydrogen, alkyl, cycloalkyl, alkenyl, heterocyclyl, (heterocyclyl)alkyl, alkynyl or aryl;
one of the hydrogen atoms of —(CH₂)ₘ— group is optionally replaced by halogen, alkyl, hydroxyl or alkoxy; and
with the proviso that R² is hydrogen when W is —(CH₂)ₘ—, wherein m is an integer of from 2 to 6;
R is hydrogen, hydroxy, alkyl, aryl, heterocyclyl, cycloalkyl or cycloalkenyl;
R⁴ is hydrogen, alkyl, alkenyl, alkynyl, aryl, cycloalkyl, heterocyclyl, amidoarylalkyl or amidoarylalkynyl;
R′ is hydrogen, aryl, alkyl or —(CH₂)ᵣ-U; wherein
r is an integer of from 1 to 4; and
U is alkenyl, alkynyl, alkylalkenyl, alkylalkynyl, arylalkenyl, arylalkynyl, heterocyclylalkenyl or heterocyclylalkynyl; or
OR′ is replaced by hydrogen;
Y is hydrogen, halogen, cyano, alkyl, aryl, heterocyclyl, hydroxy, amino, PhSe, alkenyl, alkynyl or —NR⁹R¹⁰, wherein
R⁹ and R¹⁰ are the same as defined earlier; and
Z is oxygen, sulphur or NOR¹¹, wherein
R¹¹ is the same as defined earlier.

8. The method of claim 7, wherein the condition is selected from community-acquired pneumonia, upper and lower respiratory tract infections, skin and soft tissue infections, hospital-acquired lung infections or bone and joint infections, mastitis, catheter infection, foreign body, prosthesis infections or peptic ulcer disease.

9. The method of claim 7, wherein the condition is caused by or contributed to by one or more gram positive, gram negative or anaerobic bacteria, wherein the one or more gram positive, gram negative or anaerobic bacteria are selected from Staphylococci, Streptococci, Enterococci, Haemophilus, Moraxalla spp., Chlamydia spp., Mycoplasm, Legionella spp., Mycobacterium, Helicobacter, Clostridium, Bacteroides, Corynebacterium, Bacillus or Enterobactericeae.

10. The method of claim 9, wherein one or more gram positive, gram negative or anaerobic bacteria is a cocci.

11. The method of claim 10, wherein the cocci is drug resistant.

12. The method of claim 7, wherein the one or more compounds of Formula I is concurrently or sequentially administered with one or more additional therapeutic agents selected from benzoyl peroxide, clindamycin, telithromycin, tretinoin, vitamin E, vitamin A and its derivatives, tetracycline, isotretinoin, vitamin C, vitamin D, chaparral, dandelion root, licoric root, Echinacea, kelp, cayenine, sassafras, elder flowers, pantothenic acid, para amino benzoic acid, biotin, cholin, inositol, folic acid, calcium, magnesium, potassium, vitamin B₆, zinc, carotenoid orazelaic acid or mixtures thereof.

13. A process for preparing a compound of Formula XIII, 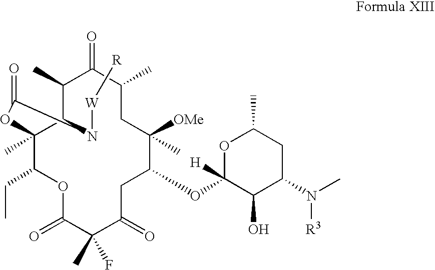
pharmaceutically acceptable salts, pharmaceutically acceptable solvates, enantiomers, diastereomers or polymorphs thereof, comprising the steps of:
(a) hydrolyzing clarithromycin of Formula II, 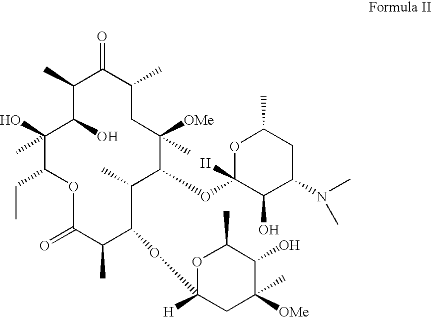
to form a compound of Formula III, 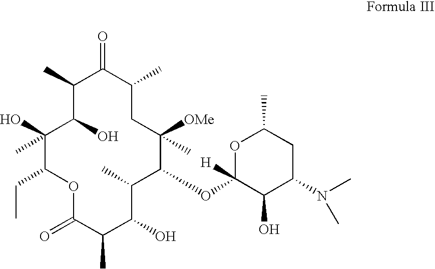
(b) protecting the compound of Formula III with one or more reagents of Formula R¹₂O or R¹X (wherein X is halogen) to form a compound of Formula IV, 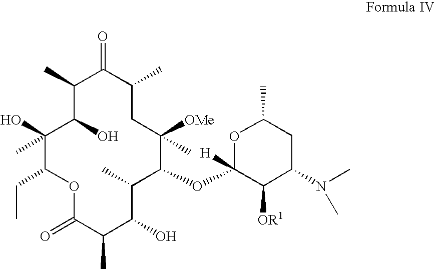
(c) reacting the compound of Formula IV with one or more suitable reagents to form a compound of Formula V, 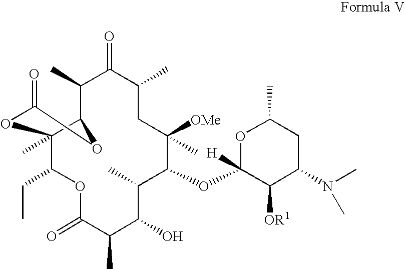
(d) reacting the compound of Formula V with one or more organic bases to form a compound of Formula VI, 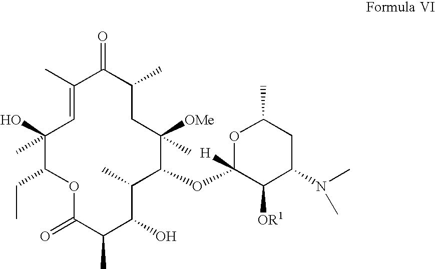
(e) oxidizing the compound of Formula VI to form a compound of Formula VII, 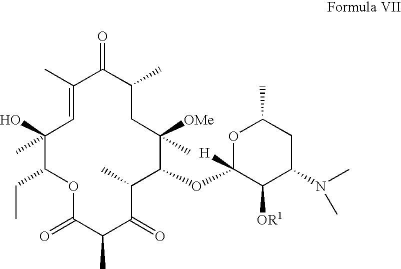
(f) desmethylating the compound of Formula VII at the 3′-N-dimethyl group to form a compound of Formula VIII, 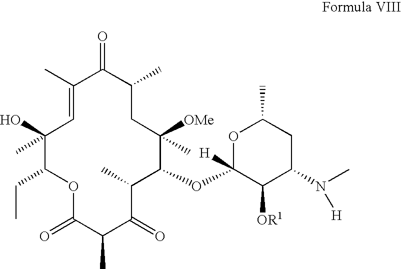
(g) alkylating the compound of Formula VIII with one or more reagents of Formula R³CHO, R³₂ CO or R³X to form a compound of Formula IX, 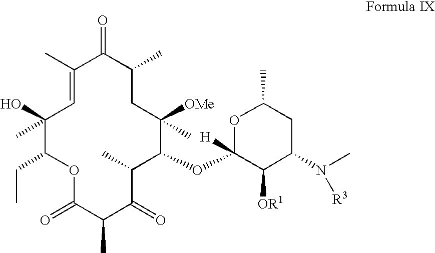
(h) fluorinating the compound of Formula IX to form a compound of Formula X, 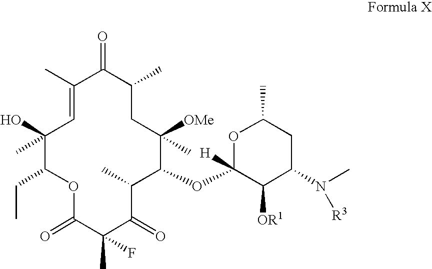
(i) reacting the compound of Formula X with N,N′-carbonyldiimidazole to form a compound of Formula XI, 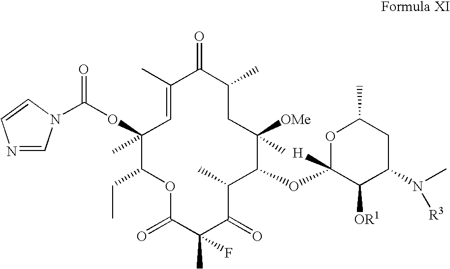
(j) reacting the compound of Formula XI with a compound of Formula R-W-NH₂ to form a compound of Formula XII, 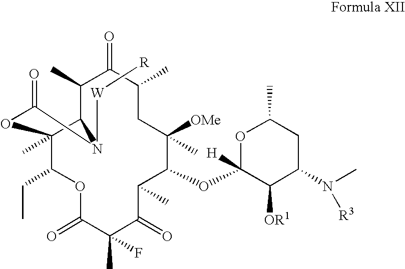
(k) deprotecting the compound of Formula XII to form a compound of Formula XIII (wherein R, R³, R¹ and W are the same as defined earlier),
wherein
R³ is hydrogen, alkyl, alkenyl, alkynyl, cycloalkyl, aryl, (heterocyclyl)alkyl or —COR¹¹, wherein
R¹¹ is hydrogen, alkyl, aryl, NR⁹R¹⁰ or alkoxy;
R⁹ and R¹⁰ are independently hydrogen, alkyl, alkenyl or alkynyl; and
with the proviso that R³ is not methyl:
W is —NH or —(CH₂)ₘ—, wherein
m is an integer of from 2 to 6;
—(CH₂)ₘ— group is optionally interrupted by one or more of unsaturated bond, oxygen, sulfur, —NRₐ— or combination thereof, wherein
Rₐ is hydrogen, alkyl, cycloalkyl, alkenyl, heterocyclyl, (heterocyclyl)alkyl, alkynyl or aryl;
one of the hydrogen atoms of —(CH₂)ₘ— group is optionally replaced by halogen, alkyl, hydroxyl or alkoxy; and
R is hydrogen, hydroxy, alkyl, aryl, heterocyclyl, cycloalkyl or cycloalkenyl; and
R¹ is hydrogen, hydroxyl protecting group.

14. A process for preparing a compound of Formula XIVA, 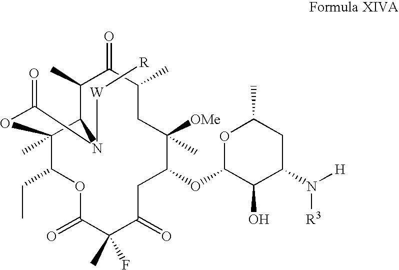
pharmaceutically acceptable salts, pharmaceutically acceptable solvates, stereoisomers, prodrugs, metabolites or polymorphs thereof comprising the steps of:
(a) desmethylating at 3′-N-dimethyl group of a compound of Formula XII 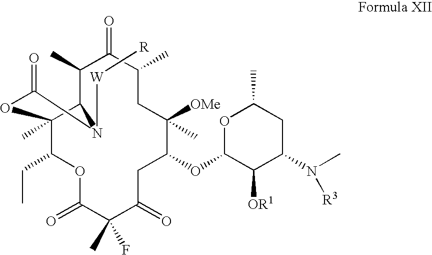
to form a compound of Formula XIIIA, 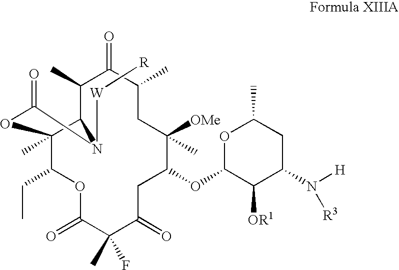
(b) deprotecting the compound of Formula XIIIA to form a compound of Formula XIVA,
wherein
R¹ is hydrogen, hydroxyl protecting group;
R³ is hydrogen, alkyl, alkenyl, alkynyl, cycloalkyl, aryl, (heterocyclyl)alkyl or —COR¹¹, wherein
R¹¹ is hydrogen, alkyl, aryl, NR⁹R¹⁰ or alkoxy;
R⁹ and R¹⁰ are independently hydrogen, alkyl, alkenyl or alkynyl; and
W is —NH or —(CH₂)ₘ—, wherein
m is an integer of from 2 to 6;
—(CH₂)ₘ— group is optionally interrupted by one or more of unsaturated bond, oxygen, sulfur, —NRₐ— or combination thereof, wherein
Rₐ is hydrogen, alkyl, cycloalkyl, alkenyl, heterocyclyl, (heterocyclyl)alkyl, alkynyl or aryl;
one of the hydrogen atoms of —(CH₂)ₘ— group is optionally replaced by halogen, alkyl, hydroxyl or alkoxy; and
R is hydrogen, hydroxy, alkyl, aryl, heterocyclyl, cycloalkyl or cycloalkenyl;

15. A process for preparing a compound of Formula XVI, 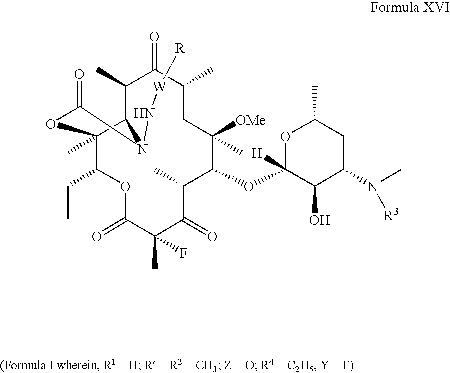
pharmaceutically acceptable salts, pharmaceutically acceptable solvates, stereoisomers, prodrugs, metabolites or polymorphs thereof, comprising the steps of:
(a) reacting the compound of Formula XI with hydrazine hydrate 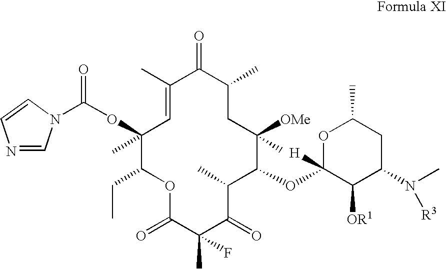
to form a compound of Formula XIV, 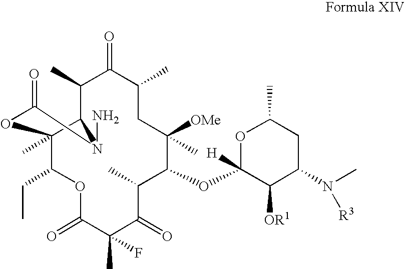
(b) deprotecting the compound of Formula XIV to form a compound of Formula XV, and 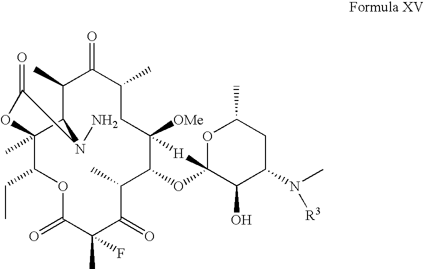
(c) reacting the compound of XV with a compound of Formula R-W-CHO to form a compound of Formula XVI,
wherein
R¹ is hydrogen, hydroxyl protecting group;
R³ is hydrogen, alkyl, alkenyl, alkynyl, cycloalkyl, aryl, (heterocyclyl)alkyl or —COR¹¹, wherein
R¹ is hydrogen, alkyl, aryl, NR⁹R¹⁰ or alkoxy;
R⁹ and R¹⁰ are independently hydrogen, alkyl, alkenyl or alkynyl; and
with the proviso that R³ is not methyl;
W is —NH or —(CH₂)ₘ—, wherein
m is an integer of from 2 to 6;
—(CH₂)ₘ— group is optionally interrupted by one or more of unsaturated bond, oxygen, sulfur, —NRₐ— or combination thereof, wherein
Rₐ is hydrogen, alkyl, cycloalkyl, alkenyl, heterocyclyl, (heterocyclyl)alkyl, alkynyl or aryl;
one of the hydrogen atoms of —(CH₂)ₘ— group is optionally replaced by halogen, alkyl, hydroxyl or alkoxy; and
R is hydrogen, hydroxy, alkyl, aryl, heterocyclyl, cycloalkyl or cycloalkenyl;

16. A process for preparing a compound of Formula XVIII, 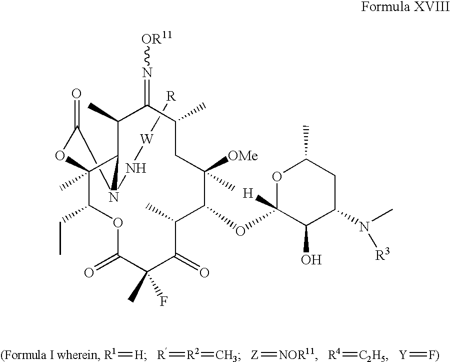
pharmaceutically acceptable salts, pharmaceutically acceptable solvates, enantiomers, diastereomers or polymorphs thereof comprising the steps of:
(a) reacting the compound of Formula XV with a compound of Formula H₂NOR¹¹ 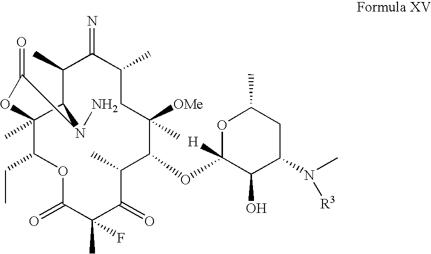
to form a compound of Formula XVII, 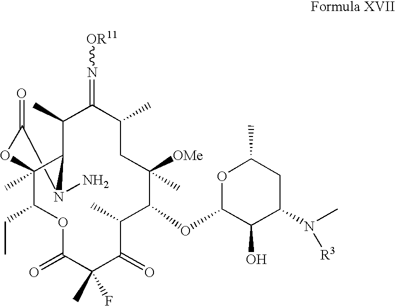
(b) reacting the compound of Formula XVII with a compound of Formula R-W-CHO to form a compound of Formula XVIII,
wherein
R¹¹ is hydrogen, alkyl, aryl, NR⁹R¹⁰ or alkoxy, wherein
R⁹ and R¹⁰ are independently hydrogen, alkyl, alkenyl or alkynyl;
R³ is hydrogen, alkyl, alkenyl, alkynyl, cycloalkyl, aryl, (heterocyclyl)alkyl or —COR¹¹, wherein
R¹¹ is the same as defined above; and
with the proviso that R³ is not methyl;
W is —NH or —(CH₂)ₘ—, wherein
m is an integer of from 2 to 6;
—(CH₂)ₘ— group is optionally interrupted by one or more of unsaturated bond, oxygen, sulfur, —NRₐ— or combination thereof, wherein
Rₐ is hydrogen, alkyl, cycloalkyl, alkenyl, heterocyclyl, (heterocyclyl)alkyl, alkynyl or aryl;
one of the hydrogen atoms of —(CH₂)ₘ— group is optionally replaced by halogen, alkyl, hydroxyl or alkoxy; and
R is hydrogen, hydroxy, alkyl, aryl, heterocyclyl, cycloalkyl or cycloalkenyl.
